(19) United States
(12) Patent Application Publication
(10) Pub. No.: US 2016/0362317 A1
(43) Pub. Date: Dec. 15, 2016

(54) METHOD OF TREATING LIQUID OR OBJECT USING GENERATION OF PLASMA IN OR NEAR LIQUID

(71) Applicant: Panasonic Intellectual Property Management Co., Ltd., Osaka (JP)

(72) Inventors: HIROKAZU KIMIYA, Kyoto (JP); SHIN-ICHI IMAI, Osaka (JP)

(21) Appl. No.: 15/171,230

(22) Filed: Jun. 2, 2016

(30) Foreign Application Priority Data
Jun. 9, 2015 (JP) 2015-117024
Jun. 9, 2015 (JP) 2015-117046

(51) Int. Cl. C02F 1/68; C02F 1/467; C02F 1/66; A61L 2/18

(52) U.S. Cl. CPC C02F 1/68; A61L 2/18; C02F 1/467; C02F 1/66; C02F 2201/461; C02F 2209/06; C02F 2303/04

(57) ABSTRACT

The method includes: preparing a plasma-treated liquid having a pH of 6 or more and 9 or less, the plasma-treated liquid being a liquid that has been treated with plasma generated in or near the liquid; and changing the pH of the plasma-treated liquid to less than 6 or to higher than 9.

### BACKGROUND

1. Technical Field

The present disclosure relates to a method of treating a liquid, a method of treating an object, a liquid treatment apparatus, an object treatment apparatus, and a plasma-treated liquid.

2. Description of the Related Art

Sterilization apparatuses utilizing plasma for cleaning and sterilizing water have been known. For example, Japanese Unexamined Patent Application Publication No. 2009-255027 discloses a sterilization apparatus for sterilizing microorganisms or bacteria with active species produced in water by means of plasma.

### SUMMARY

A method according to an aspect of the disclosure comprises: preparing a plasma-treated liquid having a pH of 6 or more and 9 or less, the plasma-treated liquid being a liquid that has been treated with plasma generated in or near the liquid; and changing the pH of the plasma-treated liquid to less than 6 or to higher than 9.

It should be noted that comprehensive or specific embodiments may be implemented as a system, a method, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating a schematic structure of a treatment liquid generation apparatus according to a First Embodiment;

FIG. 2 is a diagram illustrating an example of the structure of the treatment liquid generation apparatus according to the First Embodiment;

FIG. 3 is a flow chart showing an example of a method of generating a treatment liquid according to the First Embodiment;

FIG. 4 is a flow chart showing an example of the method of generating a treatment liquid according to the First Embodiment;

FIG. 5A is a flow chart showing a first example of the step of preparing a first treatment liquid according to the First Embodiment;

FIG. 5B is a flow chart showing a second example of the step of preparing a first treatment liquid according to the First Embodiment;

FIG. 6A is a flow chart showing a third example of the step of preparing a first treatment liquid according to the First Embodiment;

FIG. 6B is a flow chart showing a fourth example of the step of preparing a first treatment liquid according to the First Embodiment;

FIG. 7 is a flow chart showing an example of a method of treating an object according to the First Embodiment;

FIG. 8A is a graph showing the results of a test of indigo carmine decomposition by the liquid samples according to Examples 1 and 2 and Comparative Examples 1 to 3;

FIG. 8B is a graph showing the results of a test of indigo carmine decomposition by the liquid samples according to Comparative Examples 4 to 6;

FIG. 9A is a graph showing the results of a test of indigo carmine decomposition by the liquid samples according to Example 3 and Reference Examples;

FIG. 9B is a graph showing the results of a test of indigo carmine decomposition by liquid samples according to other Examples;

FIG. 9C is a graph showing the results of a test of indigo carmine decomposition by the liquid samples according to Examples 4 and 5;

FIG. 10A is a graph showing the results of a test of indigo carmine decomposition by liquid samples prepared by leaving the liquid sample according to Example 1 to stand for predetermined periods of time after the plasma treatment until the acidification;

FIG. 10B is a graph showing the results of a test of indigo carmine decomposition by liquid samples prepared by leaving the liquid sample according to Example 2 to stand for predetermined periods of time after the plasma treatment until the acidification;

FIG. 11A is a graph showing the results of a test of indigo carmine decomposition by liquid samples prepared by leaving the liquid sample according to Example 1 for predetermined periods of time;

FIG. 11B is a graph showing the results of a test of indigo carmine decomposition by liquid samples prepared by leaving the liquid sample according to Example 2 for predetermined periods of time;

FIG. 11C is a graph showing the results of a test of indigo carmine decomposition by liquid samples prepared by leaving the liquid sample according to Comparative Example 1 for predetermined periods of time;

FIG. 12A is a graph showing the results of a test of indigo carmine decomposition by liquid samples according to other Examples and Reference Examples;

FIG. 12B is a graph showing the results of a test of indigo carmine decomposition by liquid samples according to other Examples and Reference Examples;

FIG. 13A is a graph showing a relationship between the pH of the liquid samples shown in FIGS. 12A and 12B and the decomposition rates of indigo carmine;

FIG. 13B is a graph explaining the decomposition rates shown in FIG. 13A;

FIG. 14A is a graph showing the results of a test of indigo carmine decomposition by the liquid samples according to Examples 6 and 7;

FIG. 14B is a graph showing the results of a test of indigo carmine decomposition by liquid samples according to other Examples;

FIG. 15A is a graph showing the results of a test of indigo carmine decomposition by the liquid samples according to Examples 8 and 9;

FIG. 15B is a graph showing the results of a test of indigo carmine decomposition by other liquid samples according to Examples 8 and 9;

FIG. 16 is a graph showing various examples of the relationship between the dilution ratio and the decomposition time of the liquid samples according to Examples 10 to 13;

FIG. 17 is a diagram illustrating an example of the structure of a treatment liquid generation apparatus according to a Second Embodiment;

FIG. 18 is a graph showing the results of a test of indigo carmine decomposition by the liquid samples according to Example 14 and Reference Example;

FIG. 19 is a diagram illustrating an example of the structure of a treatment liquid generation apparatus according to a Third Embodiment;

FIG. 20 is a flow chart showing a method of treating an object according to the Third Embodiment;

FIG. 21 is a diagram illustrating a schematic structure of an object treatment apparatus according to a Fifth Embodiment;

FIG. 22 is a diagram illustrating an example of the structure of the object treatment apparatus according to the Fifth Embodiment;

FIG. 23 is a flow chart showing an example of the method of treating an object according to the Fifth Embodiment;

FIG. 24 is a flow chart showing another example of the method of treating an object according to the Fifth Embodiment;

FIG. 25 is a graph showing the results of a test of indigo carmine decomposition by the liquid sample according to Example 17;

FIG. 26 is a graph showing the results of a test of indigo carmine decomposition by the liquid sample according to Example 18;

FIG. 27 is a graph showing the results of a test of indigo carmine decomposition by the liquid sample according to Example 17;

FIG. 28 is a graph showing the results of a test of indigo carmine decomposition by the liquid sample according to Example 18;

FIG. 29 is a graph showing the results of a test of indigo carmine decomposition by the liquid sample according to Example 19;

FIG. 30 is a graph showing the results of a test of indigo carmine decomposition by the liquid sample according to Reference Example;

FIG. 31A is a graph showing the results of a test of indigo carmine decomposition by liquid samples according to Modification Example 1;

FIG. 31B is a graph showing the results of a test of indigo carmine decomposition by other liquid samples according to Modification Example 1; and

FIG. 32 is a graph showing the results of a test of indigo carmine decomposition by liquid samples according to Modification Example 3.

### DETAILED DESCRIPTION

### Definition of Terms

The term “neutral” means that the pH (hydrogen ion exponent) is 6 or more and 9 or less; the term “alkaline” means that the pH is higher than 9; and the term “acidic” means that the pH is less than 6.

The term “neutralization” means that the pH is adjusted to 6 or more and 9 or less; the term “alkalinization” means that the pH is adjusted to higher than 9; and the term “acidification” means that the pH is adjusted to less than 6.

The term “plasma treatment” means bringing of plasma into contact with a liquid or bringing of a gas containing active species produced by means of plasma into contact with a liquid.

The term “liquid to be plasma-treated” refers to a liquid before treatment with plasma.

The term “plasma-treated liquid” refers to a liquid after treatment with plasma. The plasma-treated liquid, for example, can function as a treatment liquid for decomposing and/or sterilizing an object. For simplification of explanation, a neutral plasma-treated liquid may be called a first treatment liquid, and a plasma-treated liquid after adjustment of the pH to acidic or alkaline may be called a second treatment liquid.

The term “method of treating a liquid” refers to a method of treating a liquid with plasma and/or changing the pH of the liquid. When a liquid subjected to the method of treating a liquid is utilized as a treatment liquid for decomposing and/or sterilizing an object, the method of treating a liquid may be called a method of generating a treatment liquid. That is, the “method of generating a treatment liquid” is an example of the method of treating a liquid. Similarly, a “treatment liquid generation apparatus” is an example of a liquid treatment apparatus.

The term “object” refers to a material to be decomposed and/or sterilized with a plasma-treated liquid.

The term “preparing a liquid” refers to not only generating a liquid but also procuring of a liquid.

The term “near a liquid” refers to a region apart from the liquid surface in an area where the active species produced by means of plasma can come into contact with liquid, for example, a region within a distance of 2 cm from the liquid surface.

The term “adding A to B” means not only that A and B are mixed by supplying A to B but also that A and B are mixed by supplying B to A, unless specifically mentioned.

### Overview of Embodiments

A method of generating a treatment liquid according to an embodiment of the present disclosure comprises: generating plasma in or near a liquid to prepare a first treatment liquid having a pH of 6 or more and 9 or less; and adjusting the pH of the first treatment liquid to generate a second treatment liquid having a pH of less than 6 or of higher than 9.

The second treatment liquid generated by acidifying or alkalinizing a neutral first treatment liquid has a high activity and excellent durability of the activity. Accordingly, the second treatment liquid can be used for, for example, decomposing and/or sterilizing an object, such as an organic material, a microorganism, or a bacterium. In addition, the neutral first treatment liquid has excellent storage stability. Accordingly, a second treatment liquid having a high activity can be generated by storing a first treatment liquid in a neutral state for a long time and then acidifying or alkalinizing the first treatment liquid. That is, the second treatment liquid generated after storage for a long time can decompose and/or sterilize an object.

For example, the first treatment liquid may be generated by adjusting the pH of a liquid to 6 or more and 9 or less during the generation of plasma in or near the liquid.

In such a case, the first treatment liquid can be prepared within a short period of time.

For example, the first treatment liquid may be generated by adjusting the pH of a liquid to 6 or more and 9 or less after the generation of plasma in or near the liquid.

In such a case, for example, even if no means for controlling the pH during the plasma treatment is provided, the first treatment liquid can be prepared simply and easily.

For example, the second treatment liquid may be generated by adding, to the first treatment liquid, (i) an acid, base, or salt; (ii) a solution containing at least one of acids, bases, and salts; (iii) a gas or solid that can be dissolved in the first treatment liquid to become an acid or a base; or (iv) a solution containing microorganisms producing the gas or the solid.

In such a case, the second treatment liquid can be readily generated. The material in the generation of a second treatment liquid from a first treatment liquid can be selected from a large number of materials. Accordingly, for example, the cost can be reduced by selecting an inexpensive material.

For example, the second treatment liquid generated by adjustment of the pH of the first treatment liquid may have a pH of less than 3.5 or higher than 10.5.

In such a case, the second treatment liquid can have a further higher activity.

For example, the first treatment liquid may be further diluted before adjustment of the pH of the first treatment liquid.

In such a case, the amount of the second treatment liquid can be increased. In addition, since the activity of the second treatment liquid may be decreased, for example, the viable cell rate or the survival rate of an object can be readily controlled.

The method of treating an object according to an embodiment of the present disclosure includes: one of the above-described methods of generating a treatment liquid; and bringing the generated second treatment liquid into contact with an object.

The second treatment liquid has a high activity and can therefore efficiently decompose and/or sterilize the object. Accordingly, for example, the time necessary for sterilizing microorganisms or bacteria can be shortened.

The method of treating an object according to an embodiment of the present disclosure includes: one of the above-described methods of generating a treatment liquid; bringing the first treatment liquid into contact with an object; and adjusting the pH of the first treatment liquid in the state that the first treatment liquid and the object are in contact with each other.

Even in such a case, the generated second treatment liquid has a high activity. The contact of the second treatment liquid and the object may be performed by any procedure. Accordingly, the first treatment liquid and/or the second treatment liquid can be used as a highly versatile treatment liquid.

The method of treating an object according to an embodiment of the present disclosure includes: one of the above-described methods of generating a treatment liquid; and adjusting the pH of the first treatment liquid concurrently with the bringing the first treatment liquid into contact with the object.

In such a case, the second treatment liquid can be brought into contact with an object concurrently with the generation of the second treatment liquid.

The treatment liquid according to an embodiment of the present disclosure is the second treatment liquid generated by the method of generating a treatment liquid.

The second treatment liquid has a high activity and can efficiently decompose and/or sterilize the object, such as microorganisms or bacteria.

The treatment liquid generation apparatus according to an embodiment of the present disclosure includes: a container for containing a liquid; a feeder for supplying a pH regulator to the container for adjusting the pH of the liquid in the container; and a control circuit for controlling the feeder. When the container contains a first treatment liquid having a pH of 6 or more and 9 or less generated by means of plasma generated in or near the liquid, the control circuit instructs the feeder to supply the pH regulator to adjust the pH of the first treatment liquid in the container to generate a second treatment liquid having a pH of less than 6 or of higher than 9.

The generated second treatment liquid has a high activity and excellent durability of the activity. Accordingly, the second treatment liquid can be used for, for example, decomposing and/or sterilizing an object, such as microorganisms or bacteria. The neutral first treatment liquid has excellent storage stability. Accordingly, a second treatment liquid having a high activity can be generated by acidifying or alkalinizing a first treatment liquid stored for a long time. That is, the second treatment liquid generated after storage for a long time can decompose and/or sterilize an object. In addition, for example, even if the first treatment liquid is generated at a place apart from the plasma generator, the activity can be preserved.

For example, the treatment liquid generation apparatus may include a plasma generator including at least one electrode pair and a power supply for applying a voltage to the electrode pair and generating plasma in or near the liquid in the container. The control circuit may instruct the plasma generator to start the generation of plasma and stop the generation of plasma after the elapse of a predetermined time to generate the first treatment liquid in the container.

In such a case, the first treatment liquid can be prepared. For example, the treatment liquid generation apparatus may include a sensor for detecting the pH of the liquid in the container and/or a feedback circuit for feedback of the pH detection result to the control circuit. This allows the first treatment liquid to be generated at a low cost.

For example, the treatment liquid generation apparatus may include a plasma generator including at least one electrode pair and a power supply for applying a voltage to the electrode pair and generating plasma in or near the liquid in the container. The control circuit may instruct the plasma generator to start the generation of plasma and to stop the generation of plasma after the elapse of a predetermined time, and then instruct the feeder to supply a pH regulator to adjust the pH of the liquid in the container to generate the first treatment liquid in the container.

In such a case, for example, the first treatment liquid can be readily prepared without controlling the duration of the plasma treatment.

For example, the treatment liquid generation apparatus may include a plasma generator including at least one electrode pair and a power supply for applying a voltage to the electrode pair and generating plasma in or near the liquid in the container. The control circuit may instruct the plasma generator to start the generation of plasma, and then (i) when the liquid in the container has an average pH per unit time of 6 or more and 9 or less, the generation of plasma is stopped after the elapse of a predetermined time to generate the first treatment liquid in the container or (ii) when the liquid in the container has an average pH per unit time of less than 6 or of higher than 9, the feeder supplies a pH regulator to adjust the pH of the liquid in the container to 6 or more and 9 or less, and then the generation of plasma is stopped after the elapse of a predetermined time to generate the first treatment liquid in the container.

In such a case, the time for contacting plasma with a neutral liquid in a container can be increased. As a result, the activity of a second treatment liquid can be enhanced.

For example, the control circuit may instruct the second treatment liquid to be discharged to the outside of the container and to be brought into contact with an object.

The second treatment liquid has a high activity and can efficiently decompose and/or sterilize an object. Accordingly, for example, the time necessary for sterilizing microorganisms or bacteria can be shortened.

For example, the control circuit may instruct the first treatment liquid to be brought into contact with an object and instruct the feeder to supply a pH regulator in a state that the first treatment liquid and the object are in contact with each other to generate the second treatment liquid.

Even in such a case, the generated second treatment liquid has a high activity. The contact of the second treatment liquid and the object may be performed by any procedure. Accordingly, the first treatment liquid and/or the second treatment liquid can be used as a highly versatile treatment liquid.

The treatment liquid according to an embodiment of the present disclosure is generated by generating plasma in or near the liquid, has a pH of 6 or more and 9 or less, and has a decomposition rate of indigo carmine of 0.02 ppm/min or less, calculated based on a change in the absorbance of light having a wavelength of 610 nm, when 10 ppm of indigo carmine is added to the liquid at 20° C. In addition, (i) when a 4.5 N sulfuric acid solution is mixed with the treatment liquid to give a pH of 2.5, the decomposition rate of indigo carmine at 10 seconds after the addition of the sulfuric acid is 0.05 ppm/min or more, or (ii) when an aqueous 4.5 N sodium hydroxide solution is mixed with the plasma-treated liquid to give a pH of 11.5, the decomposition rate of indigo carmine at 10 seconds after the addition of the aqueous sodium hydroxide solution is 0.1 ppm/min or more.

The second treatment liquid has a high activity (e.g., decomposition ability) and can efficiently decompose and/or sterilize an object, such as microorganisms or bacteria.

The method of treating an object according to an embodiment of the present disclosure includes: applying, to an object, a plasma-treated liquid generated by generating plasma in or near the liquid; and adjusting the pH of the remaining liquid after the application of the plasma-treated liquid to the object to 6 or more and 9 or less.

As a result, the remaining liquid is prevented from acting on the object. Since the activity of the remaining liquid is suppressed, for example, the remaining liquid can be safely discarded. Incidentally, the activity is an ability to cause, for example, a chemical reaction, such as oxidation or decomposition. The reduced activity of the remaining liquid can be reactivated. Accordingly, for example, the remaining liquid can be reused by reactivating the liquid. Since the activity can be reduced and reactivated, the liquid can not only decompose and/or sterilize an object, but also perform, for example, generation of a polymer by radical polymerization at high accuracy.

For example, the pH of the remaining liquid may be adjusted to 6 or more and 9 or less by adding a solution containing an acid, base, or salt to the remaining liquid.

In such a case, the pH of the remaining liquid can be readily adjusted. The acid, base, or salt can be selected from a large number of materials. For example, selection of an inexpensive material can reduce the cost.

For example, the pH of the remaining liquid may be adjusted to 6 or more and 9 or less and may be then adjusted to less than 6 or to higher than 10.

In such a case, the remaining liquid can be reactivated and thereby can be reused.

For example, the pH of the remaining liquid is adjusted to 6 or more and 9 or less, and a solution containing an acid, base, or salt may be then added to the liquid to adjust the pH to less than 6 or to higher than 10.

In such a case, the pH of the remaining liquid can be readily adjusted. The acid, base, or salt can be selected from a large number of materials. For example, selection of an inexpensive material can reduce the cost.

For example, the remaining liquid having a pH adjusted to 6 or more and 9 or less may be diluted.

In such a case, the activity can be further reduced.

The object treatment apparatus according to an embodiment of the present disclosure includes: a container for containing the plasma-treated liquid generated by generating plasma in or near a liquid; a first feeder for supplying a pH regulator to the container to adjust the pH of the liquid in the container; and a control circuit for controlling the first feeder. When the liquid remains in the container after application of the plasma-treated liquid to an object, the control circuit instructs the first feeder to supply the pH regulator to the container to adjust the pH of the remaining liquid to 6 or more and 9 or less.

As a result, the remaining liquid is prevented from acting on the object. Since the activity of the remaining liquid is suppressed, the remaining liquid can be safely discarded. The reduced activity of the remaining liquid can be reactivated. Accordingly, the remaining liquid can be reused.

For example, the pH regulator may be a solution containing an acid, base, or salt.

In such a case, the pH of the remaining liquid can be readily adjusted. The acid, base, or salt can be selected from a large number of materials. For example, selection of an inexpensive material can reduce the cost.

For example, the object treatment apparatus may further include a second feeder for supplying a dilution liquid to the container. The control circuit may instruct the second feeder to supply the dilution liquid to the container to dilute the remaining liquid after the adjustment the pH of the remaining liquid to 6 or more and 9 or less.

In such a case, the activity can be further suppressed.

Embodiments will now be specifically described with reference to the drawings.

Incidentally, the embodiments described below all show comprehensive or specific examples. The numbers, shapes, materials, components, the arrangement configuration and connection configuration of the components, steps, the order of the steps, etc. shown in the following embodiments are merely examples and are not intended to limit the present disclosure. Among the components in the following embodiments, components that are not mentioned in any independent claim describing the broadest concept will be described as optional components. In the embodiments, the method of generating a treatment liquid will be described as an example of operation of the treatment liquid generation apparatus, but is not limited to a specific apparatus structure.

### First Embodiment

### 1. Treatment Liquid Generation Apparatus

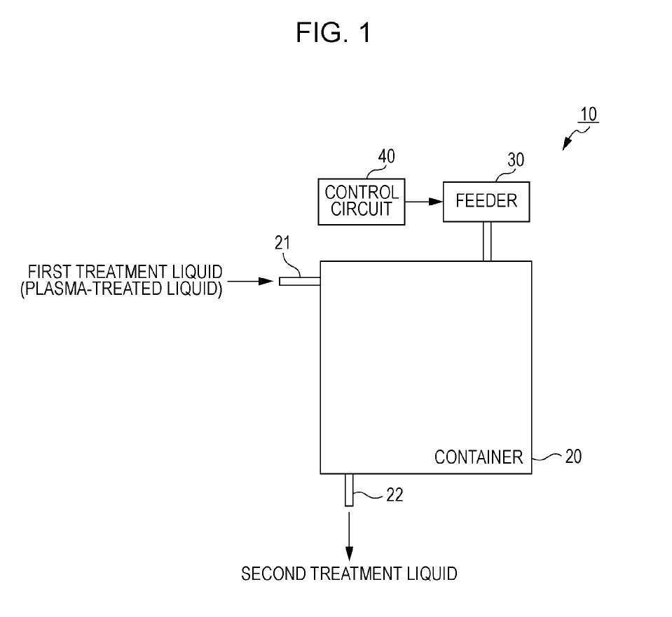

The outline of the treatment liquid generation apparatus according to a First Embodiment will be described referring to FIG. 1. FIG. 1 shows an example of the schematic structure of a treatment liquid generation apparatus 10 according to the First Embodiment.

The treatment liquid generation apparatus 10 adjusts the pH of a neutral first treatment liquid generated by generating plasma in or near the liquid to generate an acidic or alkaline second treatment liquid. As shown in FIG. 1, the treatment liquid generation apparatus 10 includes a container 20, a feeder 30, and a control circuit 40.

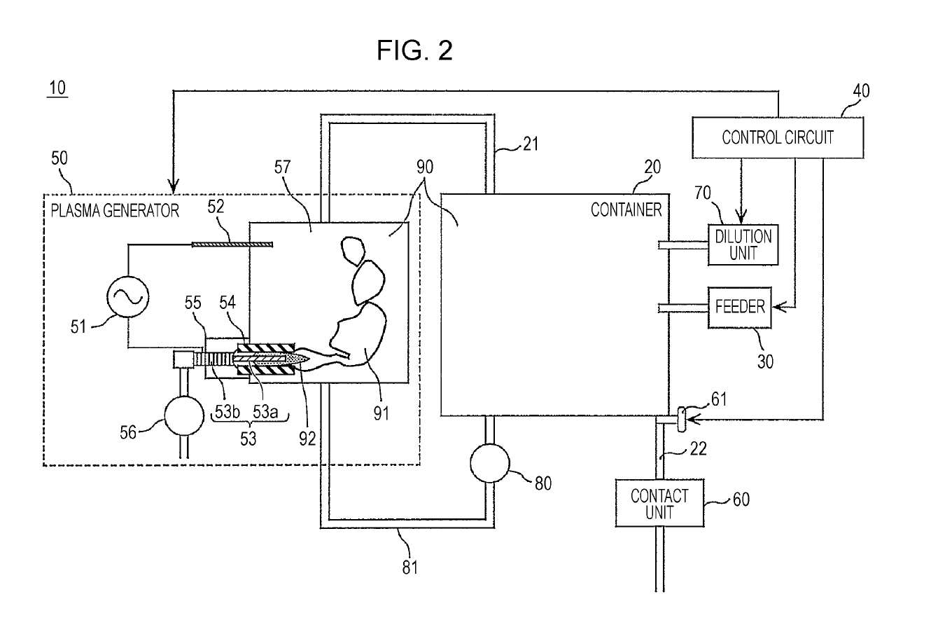

FIG. 2 shows the detailed structure of the treatment liquid generation apparatus 10 according to the First Embodiment.

As shown in FIG. 2, the treatment liquid generation apparatus 10 further includes a plasma generator 50, a contact unit 60, a valve 61, a dilution unit 70, a circulation pump 80, and a pipe 81. In the container 20, the pipe 81, and the reaction tank 57 of the plasma generator 50, a certain liquid 90 is contained.

### [1-1. Container]

The container 20 is for containing a liquid. The container 20 is provided with an inlet 21 and an outlet 22.

The container 20 is made of, for example, a material resistant to acid or alkali. For example, the container 20 is formed from a resin material, such as polyvinyl chloride or tetrafluoroethylene (PFA), a metal material, such as stainless steel, or a ceramic. The container 20 may have any size and any shape.

A neutral first treatment liquid is supplied into the container 20 through the inlet 21. The first treatment liquid is a plasma-treated liquid. The first treatment liquid may be prepared by generating plasma in a liquid (to be plasma-treated) and thereby bringing the generated plasma into contact with the liquid. Alternatively, the first treatment liquid may be prepared by generating plasma near a liquid (to be plasma-treated) and thereby bringing a gas containing active species, produced by the plasma, into contact with the liquid. In the latter case, the plasma and the liquid may not be brought into direct contact with each other.

The liquid to be plasma-treated is, for example, water, such as tap water or pure water. Alternatively, the liquid to be plasma-treated may be an alkaline solution. The liquid to be plasma-treated may be, for example, a buffer solution, such as a phosphate buffer solution, or an aqueous alkaline solution, such as an aqueous sodium hydroxide solution. If the liquid to be plasma-treated is a buffer solution, the pH can be gently changed and can be readily adjusted to a desired level.

### [1-2. Feeder and Control Circuit]

The feeder 30 supplies, to the container 20, a pH regulator for adjusting the pH of the liquid in the container 20. The feeder 30 supplies, for example, a predetermined amount of a pH regulator to the container 20 with a predetermined timing on the basis of the instruction from the control circuit 40. The feeder 30 adds, for example, a solution containing an acid, base, or salt as a pH regulator to the first treatment liquid to adjust the pH of the first treatment liquid.

The feeder 30 includes, for example, a container for containing a pH regulator, a pump, and a valve, connected to the container, for supplying the pH regulator to the container 20. For example, the control circuit 40 controls the pump to regulate the pressure difference between the container containing the pH regulator and the container 20 containing a liquid. For example, the control circuit 40 controls the switching operation of the valve.

The pH regulator is, for example, sulfuric acid (H₂SO₄), nitric acid (HNO₃), an aqueous sodium hydroxide (NaOH) solution, an aqueous ammonia (NH₃) solution, or a salt such as aluminum sulfate (Al₂(SO₄)₃) or magnesium chloride (MgCl₂). These pH regulators are merely examples, and the pH regulator may be in any form, such as a solid, liquid, or gas, as long as the material can adjust the pH of a liquid. For example, the pH regulator may be a microorganism that produces a material capable of adjusting the pH of a liquid.

The control circuit 40 controls the feeder 30. For example, the control circuit 40 instructs the feeder 30 to supply a pH regulator to the container 20 when a first treatment liquid is contained in the container 20. As a result, for example, the pH of the neutral first treatment liquid is adjusted to generate an acidic or alkaline second treatment liquid in the container 20. The second treatment liquid is discharged to the outside from the outlet 22 of the container 20, as necessary. The discharged second treatment liquid is used for, for example, decomposition and/or sterilization of an object.

The control circuit 40 may control the amount of the pH regulator to be supplied from the feeder 30 to the container 20 to generate a strong acidic or alkaline second treatment liquid from a neutral first treatment liquid.

The control circuit 40 includes, for example, a non-volatile memory storing a program and a processor executing the program. The control circuit 40 may further include a volatile memory, which is a temporary storage area for executing the program, and input and output ports. The control circuit 40 is, for example, a microcomputer.

### [1-3. Plasma Generator and Control Circuit]

The plasma generator 50 generates plasma 92 in a liquid 90. For example, the plasma generator 50 generates plasma 92 in a bubble 91 formed in the liquid 90. The bubble 91 is formed from the gas supplied by the gas feeder 56.

As shown in FIG. 2, the plasma generator 50 includes a power supply 51, a first electrode 52, a second electrode 53, an insulator 54, a holding block 55, a gas feeder 56, and a reaction tank 57. Examples of each component of the plasma generator 50 will now be described in detail.

The power supply 51 is connected between the first electrode 52 and the second electrode 53. The power supply 51 supplies a predetermined voltage between the first electrode 52 and the second electrode 53. The predetermined voltage is, for example, a pulse voltage or an AC voltage. The predetermined voltage is, for example, 1 to 50 kV with a voltage pulse of 1 to 100 kHz. The voltage waveform may be, for example, any of pulse, half sine, and sine waveforms. The value of the current flowing between the first electrode 52 and the second electrode 53 is, for example, 1 mA to 3 A. For example, the power supply 51 applies, between the first electrode 52 and the second electrode 53, a pulse voltage having a peak voltage of 4 kV, a pulse width of 1 μsec, and a frequency of 30 kHz. For example, the input power by the power supply 51 is 10 to 100 W. The input power herein is a power charged from a commercial power supply and is different from the power consumed for generating plasma. That is, the reactive power is also included in this power, and the power actually consumed for generating plasma may be less than the input power.

The first electrode 52, one of an electrode pair, is disposed so as to pass through the wall of the reaction tank 57. The first electrode 52 is at least partially in contact with the liquid 90. The first electrode 52 is, for example, a rod-like electrode. The first electrode 52 is, for example, made of a conductive metal material, such as copper, aluminum, or iron.

The second electrode 53, the other of the electrode pair, is disposed so as to pass through the wall of the reaction tank 57. The second electrode 53 is at least partially in contact with the liquid 90, at least when no power is supplied from the power supply 51. The second electrode 53 is used as a reaction electrode. When a predetermined voltage is applied between the first electrode 52 and the second electrode 53, plasma 92 is generated in the circumference of the second electrode 53. For example, the plasma 92 is generated in the bubble 91.

In the example shown in FIG. 2, the second electrode 53 includes a metal electrode portion 53a and a metal screw portion 53b.

The metal electrode portion 53a is press-inserted into the metal screw portion 53b and is unified to the metal screw portion 53b. The metal electrode portion 53a is formed so as not to protrude from the opening of the insulator 54. The metal electrode portion 53a is, for example, a rod-like electrode and is formed from a plasma-resistant metal material, such as tungsten. Alternatively, though the durability is decreased, the metal electrode portion 53a may be formed from, for example, copper, aluminum, or iron.

The metal screw portion 53b supports the press-inserted metal electrode portion 53a. The metal screw portion 53b is, for example, a rod-like member and is formed from iron. Alternatively, the metal screw portion 53b may be made of, for example, copper, zinc, aluminum, tin, or brass, instead of iron.

The metal screw portion 53b includes a screw part (e.g., male screw) that is screwed into a screw part (e.g., female screw) provided to the holding block 55. Such a structure can adjust the positional relation between the metal electrode portion 53a and the insulator 54.

The metal screw portion 53b is, for example, provided with a through-hole (not shown) passing through in the axial direction. One end of the through-hole communicates with the gap between the metal electrode portion 53a and the insulator 54. The other end of the through-hole is connected to the gas feeder 56. Accordingly, the gas supplied from the gas feeder 56 is supplied to the liquid 90 through the through-hole and the gap and thereby forms a bubble 91 in the liquid 90.

The insulator 54 is disposed so as to surround the outer surface of the metal electrode portion 53a. The insulator 54 has, for example, a cylindrical shape. The insulator 54 has an inner diameter larger than the outer diameter of the metal electrode portion 53a. Consequently, a gap is formed between the inner surface of the insulator 54 and the outer surface of the metal electrode portion 53a.

The insulator 54 may be formed from, for example, an alumina ceramic or may be formed, for example, magnesia, quartz, or yttrium oxide.

The holding block 55 is a member for supporting the metal screw portion 53b and the insulator 54. The holding block 55 is provided with a screw part (e.g., female screw). The positional relation between the holding block 55 and the metal screw portion 53b can be controlled by rotating the metal screw portion 53b around the axis. Such a structure can adjust the positional relation between the insulator 54 and the metal electrode portion 53a. For example, the front edge of the metal electrode portion 53a can be adjusted not to protrude from the opening of the insulator 54.

The gas feeder 56 supplies a gas to the liquid 90, and thereby a bubble 91 is formed in the liquid 90. The bubble 91 is discharged into the liquid 90 in the reaction tank 57 through the opening of the insulator 54. The gas feeder 56 is, for example, a pump.

The gas feeder 56 takes in, for example, the air present in the periphery of the plasma generator 50 and then supplies this air to the liquid 90 in the reaction tank 57. The gas supplied by the gas feeder 56 is not limited to air and may be any gas that can be ionized into a plasma form, such as nitrogen, oxygen, a noble gas, such as argon, or water vapor. The gas is supplied to the liquid 90 through the through-hole provided to the metal screw portion 53b and the gap between the metal electrode portion 53a and the insulator 54, and thereby the gas forms a bubble 91 in the liquid 90. The metal electrode portion 53a is, for example, covered with the bubble 91 and can be kept in a state of not being in direct contact with the liquid 90. In this state, plasma 92 can be generated in the bubble 91.

The reaction tank 57 is a container for generating plasma 92 therein. The reaction tank 57 is connected to the pipe 81. The circulation pump 80 circulates the liquid 90 between the reaction tank 57 and the container 20 through the pipe 81. The reaction tank 57 may be a part of the pipe 81.

For example, the circulation pump 80 sends the liquid 90 from the container 20 to the reaction tank 57, within which plasma 92 is generated in the liquid 90 to thereby generate a first treatment liquid. The first treatment liquid generated in the reaction tank 57 is supplied to the container 20 through the inlet 21.

The reaction tank 57 is formed from, for example, a material resistant to acid and/or alkali. For example, the reaction tank 57 is formed from a resin material, such as polyvinyl chloride or tetrafluoroethylene (PFA), a metal material, such as stainless steel, or a ceramic. The reaction tank 57 may have any size and any shape.

The reaction tank 57 and the container 20 may be unified. That is, the plasma generator 50 may not have the reaction tank 57 and may generate plasma 92 in the container 20. In such a case, the treatment liquid generation apparatus 10 may not have the circulation pump 80 and the pipe 81.

The control circuit 40 may control, for example, the plasma generator 50. The control circuit 40 controls, for example, the power supply 51 and the gas feeder 56. The control circuit 40 controls the timing and the period of applying a voltage between the first electrode 52 and the second electrode 53 by the power supply 51. That is, the control circuit 40 controls the timing of generating plasma 92 in the liquid 90 and the period of the plasma generation (i.e., the duration of the plasma treatment). In addition, the control circuit 40 controls, for example, the timing and the amount of the gas supply to the liquid 90 by the gas feeder 56.

For example, the control circuit 40 places a liquid to be plasma-treated having a predetermined pH in the container 20 and then instructs the plasma generator 50 to start generation of plasma 92 and to stop the generation of plasma 92 after the elapse of a predetermined time. Subsequently, the control circuit 40 may instruct, for example, the feeder 30 to supply a pH regulator to the container 20 to adjust the pH of the liquid 90 to 6 or more and 9 or less.

Alternatively, the control circuit 40 may start the generation of plasma 92 by the plasma generator 50 to adjust the average pH per unit time of the liquid 90 in the container 20 to 6 or more and 9 or less and to stop the generation of plasma 92 after the elapse of a predetermined time. Alternatively, the control circuit 40 may stop the generation of plasma 92 when the average pH per unit time of the liquid 90 in the container 20 reached 6 or more and 9 or less, instead of measuring the elapsed time.

In such a case, the control circuit 40 generates a neutral first treatment liquid in the container 20.

The treatment liquid generation apparatus 10 may not have the plasma generator 50. In such a case, for example, a first treatment liquid generated in advance at another place is placed in the container 20.

### [1-4. Contact Unit and Valve]

The contact unit 60 is a portion for bringing the second treatment liquid into contact with an object. The contact unit 60 is connected to, for example, the outlet 22 of the container 20 through the valve 61. The contact unit 60 may be, for example, a container for containing an object. In such a case, the second treatment liquid is placed in the container through the outlet 22 to bring the second treatment liquid into contact with the object. Alternatively, the contact unit 60 may be, for example, an injector, a spray, or a diffuser. In such a case, the second treatment liquid is sprayed toward the object to be brought into contact with the object.

The object is a material to be decomposed and/or sterilized by the second treatment liquid. The object is, for example, an organic material, a microorganism, or a bacterium. The contact unit 60 brings the second treatment liquid discharged from the outlet 22 into contact with, for example, a material containing an object. The material containing an object is, for example, daily commodities, such as tableware, medical instrument, or a building material, such as the floor or window glass of a bathroom. Alternatively, the material containing an object is, for example, the human oral cavity containing a pathogen of dental caries or periodental disease; or a food, animal, or a plant containing putrefactive bacteria.

The valve 61 is provided to the outlet 22, and the switching thereof is controlled by the control circuit 40. For example, the liquid contained in the container 20 is supplied to the contact unit 60 through the outlet 22 by opening the valve 61 and is brought into contact with an object. For example, after the generation of a second treatment liquid, the control circuit 40 opens the valve 61 to bring the second treatment liquid into contact with the object.

The treatment liquid generation apparatus 10 may bring the second treatment liquid and an object into contact with each other by means other than the contact unit 60. For example, the treatment liquid generation apparatus 10 may further include a feeder (not shown) for supplying an object to the container 20. The feeder may be an inlet provided to the container 20 for supplying an object to the container 20 by a user. The feeder may further include a container for containing an object, and the container may be connected to the inlet through a valve. In such a structure, for example, the feeder supplies the object to the container 20 to form a mixture of the object and the first treatment liquid, and the pH of the first treatment liquid (or the mixture of the first treatment liquid and the object) can be then adjusted. In such a case, generation of a second treatment liquid and contact of the second treatment liquid with the object can be concurrently performed.

For example, the treatment liquid generation apparatus 10 may include a container for containing a mixture of an object and a pH regulator. In such a structure, the mixture may be brought into contact with a first treatment liquid by supplying the mixture to the first treatment liquid or by supplying the first treatment liquid to the mixture. In both cases, the pH of the first treatment liquid is adjusted concurrently with the contact of the first treatment liquid with the object. As a result, generation of a second treatment liquid and contact of the second treatment liquid with the object can be concurrently performed. Alternatively, for example, an object and a pH regulator may be concurrently supplied to the container 20 from different containers.

### [1-5. Dilution Unit]

The dilution unit 70 dilutes the first treatment liquid. For example, the dilution unit 70 dilutes the first treatment liquid before the adjustment of the pH of the first treatment liquid. The dilution unit 70, for example, supplies a dilution liquid to the container 20. The dilution liquid may be, for example, a buffer solution having a pH equivalent to that of the first treatment liquid. Alternatively, the dilution liquid may be, for example, water such as pure water or tap water. The timing of dilution and the degree of dilution by the dilution unit 70 can be controlled by the control circuit 40. The dilution unit 70 includes, for example, a valve for controlling the inflow of the dilution liquid into the container 20. The dilution unit 70, for example, includes a container containing the dilution liquid.

### [1-6. Circulation Pump and Pipe]

The circulation pump 80 is an example of the liquid feeder provided to the pipe 81. The circulation pump 80 is, for example, a chemical pump.

The circulation pump 80 circulates the liquid 90 between the container 20 and the reaction tank 57 through the pipe 81. That is, the circulation path of the liquid 90 is composed of the container 20, the pipe 81, and the reaction tank 57.

The pipe 81 is a tube for forming the circulation path for circulating the liquid 90. The pipe 81 is formed from, for example, a tubular member, such as a pipe, tube, or hose. The pipe 81 is formed from, for example, the same material as that of the container 20.

### 2. Operation

### [2-1. Method of Generating Treatment Liquid]

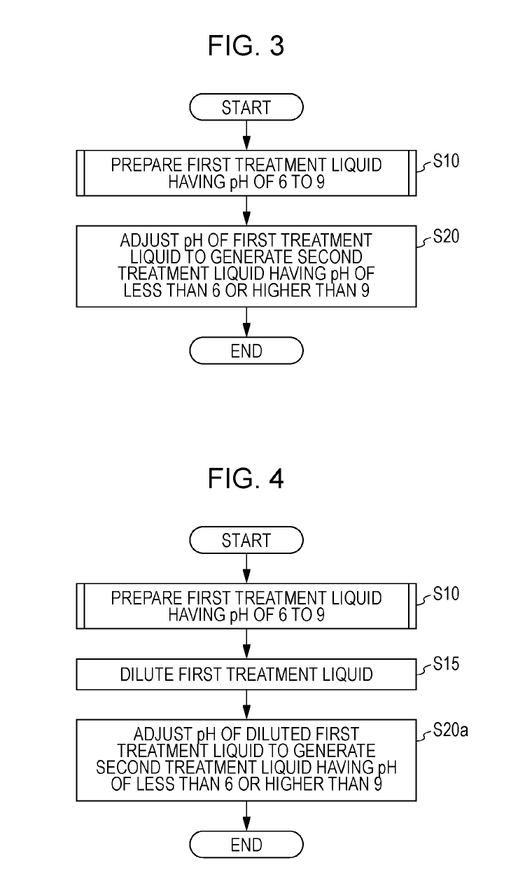

Examples of the operation of the treatment liquid generation apparatus 10 according to the Embodiment will be described using FIGS. 3 to 6B. A method of generating a treatment liquid according to the Embodiment will be described using FIGS. 3 and 4.

FIG. 3 is a flow chart showing a method of generating a treatment liquid according to the First Embodiment.

First, a first treatment liquid having a pH of 6 or more and 9 or less is prepared (S10). The prepared first treatment liquid is contained in the container 20.

Subsequently, the treatment liquid generation apparatus 10 adjusts the pH of the first treatment liquid to generate a second treatment liquid having a pH of less than 6 or of higher than 9 (S20). For example, the feeder 30 supplies a pH regulator to the container 20 based on the instruction from the control circuit 40. For example, the feeder 30 adds a solution containing an acid, base, or salt to the first treatment liquid. On this occasion, the feeder 30 may add a large amount of a pH regulator to the first treatment liquid to generate a second treatment liquid having a pH of less than 3.5 or of higher than 10.5.

As described below, the first treatment liquid has excellent storage stability. Accordingly, the method may include a storage time after step S10 and before step S20. The storage time may be a long period, such as several hours, several days, or several months.

The preparation (e.g., generation) of the first treatment liquid (S10) and the generation of the second treatment liquid (S20) are performed by different procedures. For example, the first treatment liquid is generated by plasma treatment, and the second treatment liquid is generated by adding a pH regulator to the first treatment liquid. In the generation of the second treatment liquid from the first treatment liquid, plasma treatment is not performed.

For example, the first treatment liquid is stored in a container for storage. The first treatment liquid may be discharged from the storage container and then be supplied to a reaction container. An amount of the first treatment liquid which is supplied to the reaction container may be determined based on the input from a user. A pH regulator is added to the first treatment liquid in the reaction container to generate a second treatment liquid. As a result, the generated second treatment liquid can be used for decomposition and/or sterilization of the object.

FIG. 4 is a flow chart showing another example of the method of generating a treatment liquid according to the First Embodiment. As shown in FIG. 4, the step (S10) of preparing a first treatment liquid is the same as step S10 shown in FIG. 3.

The treatment liquid generation apparatus 10 dilutes the prepared first treatment liquid (S15) before adjustment of the pH of the first treatment liquid. For example, the dilution unit 70 supplies a dilution liquid to the container 20 based on the instruction from the control circuit 40. The dilution liquid is, for example, a buffer solution having a pH equivalent to that of the first treatment liquid or water, such as pure water or tap water.

Subsequently, the pH of the diluted first treatment liquid is adjusted to generate a second treatment liquid having a pH of less than 6 or of higher than 9 (S20a). This step S20a is the same as, for example, step S20 shown in FIG. 3.

As a result, the amount of the second treatment liquid can be increased. For example, a large amount of a second treatment liquid can be generated from a small amount of a first treatment liquid. Accordingly, a large amount of an object can be treated and can be, for example, used in a broad range of sterilization treatment. Even if the second treatment liquid is generated from a diluted first treatment liquid, the second treatment liquid still has a high activity, which will be described in detail below.

### [2-2. Generation of First Treatment Liquid]

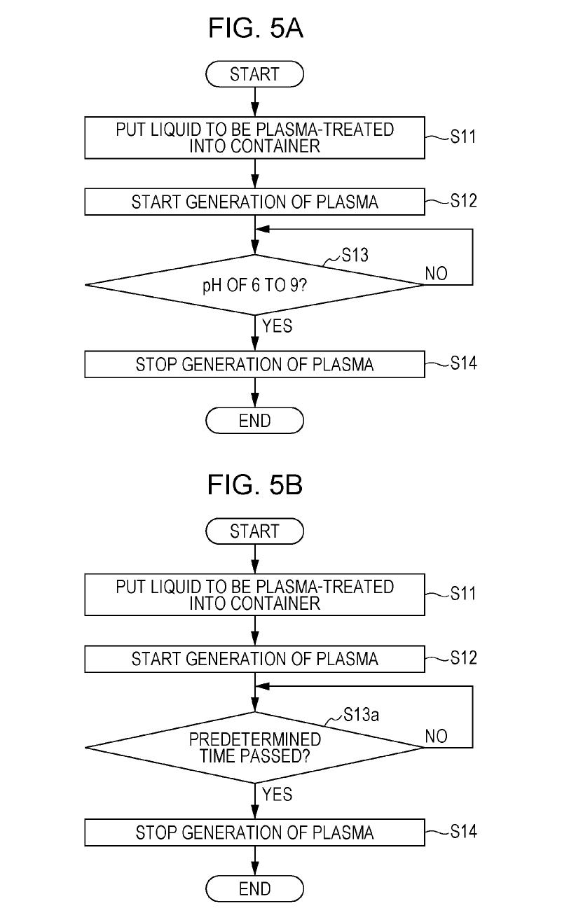

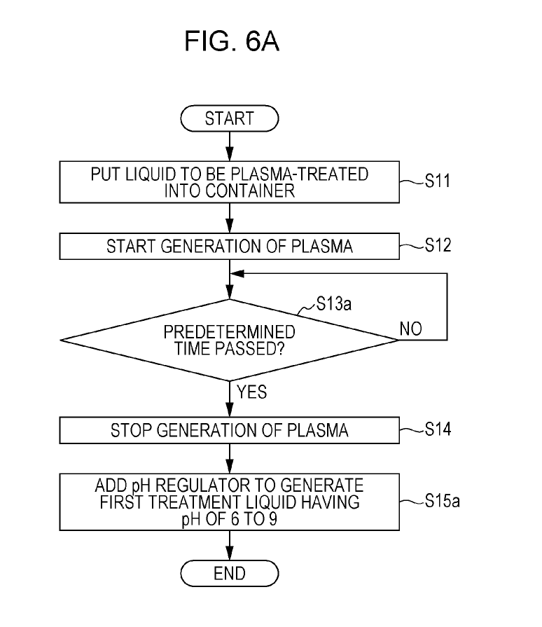

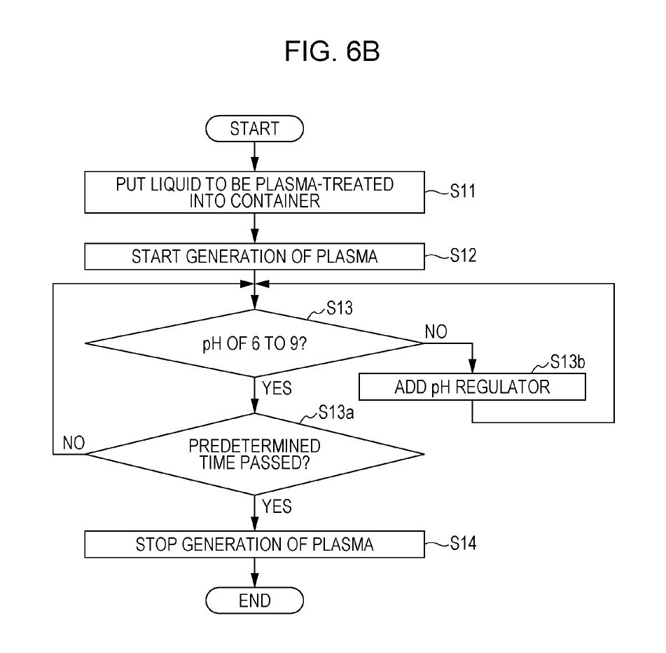

The step of preparing a neutral first treatment liquid according to the First Embodiment will now be described using FIGS. 5A to 6B. FIGS. 5A to 6B are flow charts each showing the step (S10) of preparing a first treatment liquid according to the Embodiment.

FIG. 5A is a flow chart showing a first example of the step (S10) of preparing a first treatment liquid according to the First Embodiment.

First, a liquid to be plasma-treated is placed in the container 20 (S11). The liquid to be plasma-treated is a liquid 90 not subjected to the plasma treatment and is, for example, tap water or a buffer solution. For example, the inlet 21 of the container 20 is connected to a water pipe (not shown) through a valve (not shown). The control circuit 40 controls the switching of the valve to supply a predetermined amount of tap water to the container 20.

Subsequently, generation of plasma is started (S12). For example, the gas feeder 56 supplies a gas to the liquid 90 based on the instruction from the control circuit 40. The second electrode 53 is covered with the bubble 91 of the supplied gas. In this state, the power supply 51 applies a voltage between the first electrode 52 and the second electrode 53 based on the instruction from the control circuit 40. As a result, electric discharge is caused in the bubble 91 to generate plasma 92 therein. The generated plasma 92 acts on the liquid 90 and changes the ionic composition of the liquid 90 to vary the pH of the liquid 90. Alternatively, the plasma 92 acts on the supplied gas to generate a product, and the product is dissolved in the liquid 90 to vary the pH of the liquid 90.

When the pH of the liquid 90 does not reach a predetermined value, i.e., a pH of 6 or more or 9 or less (the case of “No” in S13), the generation of plasma 92 is continued. For example, the control circuit 40 instructs the power supply 51 to continue the application of a voltage.

When the pH of the liquid 90 is 6 or more or 9 or less (the case of “Yes” in S13), the generation of plasma 92 is stopped (S14). For example, the power supply 51 stops the application of a voltage between the first electrode 52 and the second electrode 53 based on the instruction from the control circuit 40. In addition, the gas feeder 56 stops the supply of the gas based on the instruction from the control circuit 40.

The container 20 may be provided with a pH sensor for detecting the pH of the liquid 90. The control circuit 40 may receive the pH value of the liquid 90 from the pH sensor and may stop the generation of plasma 92 based on the received pH value.

The pH sensor is, for example, a glass electrode pH meter. The glass electrode pH meter uses, for example, a potassium chloride solution or an ionic liquid salt bridge as a liquid junction, and uses Ag/AgCl as electrodes. The pH sensor may be, for example, an ISFET pH meter. Alternatively, the determination of the pH may be colorimetric measurement including sampling a liquid and using a pH indicator or a pH test paper.

As described above, the treatment liquid generation apparatus 10 can generate a first treatment liquid having a pH of 6 or more and 9 or less.

The pH sensor may not be provided. In such a case, the duration of the plasma treatment may be set to an appropriate period for giving the pH of the liquid 90 in a range of 6 or more and 9 or less based on, for example, the type of the gas to be supplied by the gas feeder 56, the type and the volume of the liquid 90, and the voltage to be applied.

FIG. 5B is a flow chart showing a second example of the step (S10) of preparing a first treatment liquid according to the First Embodiment. For example, steps S11, S12, and S14 in FIG. 5B are respectively the same as steps S11, S12, and S14 in FIG. 5A.

The control circuit 40 continues the application of a voltage when the elapsed time from the start of application of the voltage has not reached a predetermined time (the case of “No” in S13a). The control circuit 40 stops the application of a voltage (S14) when the elapsed time has reached the predetermined time (the case of “Yes” in S13a). For example, the control circuit 40 includes a timer for measuring the elapsed time from the start of plasma treatment.

The duration of the plasma treatment can be set by, for example, as follows. For example, when the gas supplied by the gas feeder 56 is air, a part of nitrogen in the supplied air is oxidized to nitric acid. This nitric acid is dissolved in the liquid 90 to reduce the pH of the liquid 90. Accordingly, if the variation characteristics in the pH of the liquid are obtained in advance, a buffer solution can be prepared based on the variation characteristics. The buffer solution has a pH adjusted so as to compensate the pH variation due to, for example, the plasma treatment for a predetermined time. The use of the buffer solution as an untreated liquid 90 makes the pH of the first treatment liquid within a range of 6 or more and 9 or less after the plasma treatment for a predetermined time.

Alternatively, the duration of the plasma treatment may be set to an arbitrary time, without being set to an appropriate time. For example, when the gas supplied by the gas feeder 56 is argon and when the liquid to be plasma-treated is a buffer solution having a pH of 6 or more and 9 or less, the pH variation associated with plasma treatment is significantly small. Accordingly, even if the duration of the plasma treatment is not set to an appropriate time, a first treatment liquid having a pH of 6 or more and 9 or less can be prepared.

FIG. 6A is a flow chart showing a third example of the step (S10) of preparing a first treatment liquid according to the First Embodiment. For example, steps S11, S12, S13a, and S14 shown in FIG. 6A are respectively the same as steps S11, S12, S13a, and S14 shown in FIG. 5B.

After the stop of the generation of plasma 92 (S14), a pH regulator is added to the plasma-treated liquid to generate a first treatment liquid having a pH of 6 or more and 9 or less (S15a). For example, if the pH of the liquid 90 at the time of stopping the generation of plasma 92 is less than 6, the feeder 30 adds a solution containing a base to the liquid 90 based on the instruction from the control circuit 40. The amount of the base to be added is determined based on, for example, the amount and pH of the liquid 90.

FIG. 6B is a flow chart showing a fourth example of the step (S10) of preparing a first treatment liquid according to the First Embodiment. For example, steps S11, S12, and S14 shown in FIG. 6B are respectively the same as steps S11, S12, and S14 shown in FIG. 5A.

In the flow chart shown in FIG. 6B, when the average pH per unit time of the liquid 90 is 6 or more and 9 or less (the case of “Yes” in S13) and when the elapsed time from the start of the generation of plasma 92 has reached a predetermined time (the case of “Yes” in S13a), the generation of plasma 92 is stopped (S14). As a result, the first treatment liquid is generated.

In contrast, when the average pH per unit time of the liquid 90 is not in the range of 6 to 9 (the case of “No” in S13), the control circuit 40 instructs the feeder 30 to supply a pH regulator to the liquid (S13b). When the elapsed time from the start of the generation of plasma 92 has not reached the predetermined time (the case of “No” in S13a), the generation of plasma 92 is continued, and the step returns to step S13.

The pH value of the liquid 90 is measured by, for example, a pH sensor. In the process shown in FIG. 6B, for example, the time for allowing plasma to be in contact with the liquid can be increased while the liquid in the container being maintained in a neutral state. As a result, the activity of the second treatment liquid can be enhanced.

The pH regulator for generating the first treatment liquid may be different from that for generating a second treatment liquid from the first treatment liquid. For example, the pH regulator for generating the first treatment liquid may be a solution containing a base or salt, a neutral buffer solution, or a combination thereof. As a result, the acidic liquid having a reduced pH due to the plasma treatment can be neutralized. In contrast, the pH regulator for generating a second treatment liquid may be a solution containing an acid for acidifying the neutral liquid or may be a solution containing a base or salt for alkalinizing the neutral liquid.

### [2-3. Method of Treating Object]

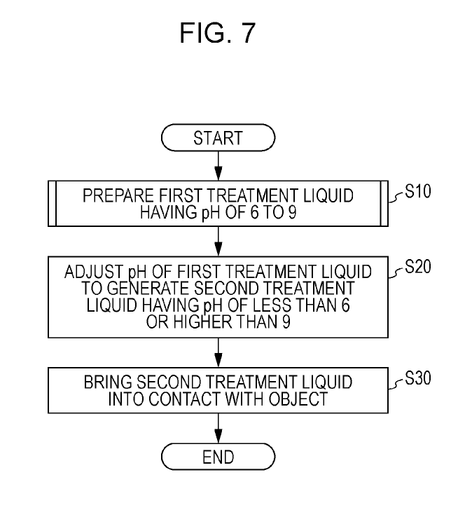

A method utilizing a second treatment liquid for treating an object will now be described using FIG. 7.

FIG. 7 is a flow chart showing a method of treating an object according to the First Embodiment. As shown in FIG. 7, the steps, S10 and S20, until the generation of a second treatment liquid are respectively the same as steps S10 and S20 shown in FIG. 3, for example.

The treatment liquid generation apparatus 10 generates a second treatment liquid and then brings the generated second treatment liquid into contact with an object (S30). For example, the control circuit 40 opens the valve 61 and thereby supplies the second treatment liquid from the container 20 to the contact unit 60 through the outlet 22. The contact unit 60 brings the supplied second treatment liquid into contact with the object.

Steps S10 and S20 may be concurrently performed. For example, the object may be mixed with a pH regulator in advance. In such a case, a mixture of the object and the pH regulator is further mixed with a first treatment liquid. Alternatively, the object, the pH regulator, and the first treatment liquid may be simultaneously mixed. In such a case, the contact of the second treatment liquid with the object can be performed concurrently with the generation of the second treatment liquid.

### 3. Examples (Corresponding to FIG. 5B)

A variety of examples of the treatment liquid generation apparatus 10 according to the First Embodiment will now be described using drawings. The present inventors generated the following liquid samples according to Examples 1 to 6 and Comparative Examples 1 to 3 and performed a test of indigo carmine decomposition by these liquid samples.

### [3-1. Conditions]

In Examples 1 to 5 and Comparative Example 1, the treatment liquid generation apparatus 10 shown in FIG. 2 was used for the plasma treatment of a liquid to be plasma-treated contained in the container 20. The container 20 was made of PFA and contained 100 mL of a liquid 90. The container 20 was provided with a pH sensor, and the pH and temperature of the liquid 90 were monitored at all times.

The circulation pump 80 was a chemical pump, and the flow rate in the pipe 81 was adjusted to 0.6 L/min. The gas feeder 56 supplied air at 0.3 L/min to the liquid 90. The power supply 51 supplied a power of 20 W for 30 minutes. That is, the time for generating plasma 92, i.e., the duration of plasma treatment, was 30 minutes.

The conditions of each Example and Comparative Example will now be described in detail. Table 1 summarizes the conditions of each Example, and Table 2 summarizes the conditions of each Comparative Example.

TABLE 1

| | Example 1 | Example 2 | Example 3 | Example 4 | Example 5 |
|---|---|---|---|---|---|
| Liquid to be plasma- | Phosphate | Phosphate | Phosphate | NaOH solution | NaOH solution |
| treated and pH | buffer solution | buffer solution | buffer solution | pH 12 | pH 12 |
| | pH 8.3 | pH 8.3 | pH 8.3 | | |
| Plasma treatment | Discharge in a | Discharge in a | Discharge in a | Discharge in a | Discharge in a |
| | bubble in liquid | bubble in liquid | bubble in liquid | bubble in liquid | bubble in liquid |
| pH of first treatment | pH 7 | pH 7 | pH 7 | pH 7 | pH 7 |
| liquid | | | | | |
| pH adjustment | Addition of | Addition of | Addition of | Addition of | Addition of |
| procedure | sulfuric acid | NaOH | Al₂(SO₄)₃ | sulfuric acid | NaOH |
| pH of second | pH 2.71 | pH 11.6 | pH 2.5 | pH 2.5 | pH 11.5 |
| treatment liquid | | | | | |

In Example 1, a 10 mM phosphate buffer solution having a pH of 8.3 was used as the liquid to be plasma-treated (i.e., liquid 90). This phosphate buffer solution was prepared by mixing 37 mg of sodium dihydrogen phosphate dihydrate and 683 mg of disodium hydrogen phosphate, and the mixture was diluted with ultra-pure water to 500 mL in a measuring cylinder. The first treatment liquid after the plasma treatment had a pH of 7. A 4.5 N sulfuric acid solution was added to the first treatment liquid to generate a second treatment liquid having a pH of 2.71. That is, in Example 1, a buffer solution was plasma-treated while maintaining the neutrality, and the plasma-treated buffer solution was then acidified by adding an acid.

In Example 2, the same phosphate buffer solution having a pH of 8.3 as that in Example 1 was used as the liquid to be plasma-treated. The first treatment liquid after the plasma treatment had a pH of 7. An aqueous 4.5 N sodium hydroxide solution was added to the first treatment liquid to generate a second treatment liquid having a pH of 11.6. That is, in Example 2, a buffer solution was plasma-treated while maintaining the neutrality, and the plasma-treated buffer solution was then alkalinized by adding a base.

In Example 3, the same phosphate buffer solution having a pH of 8.3 as that in Example 1 was used as the liquid to be plasma-treated. The first treatment liquid after the plasma treatment had a pH of 7. Aluminum sulfate was added to the first treatment liquid to generate a second treatment liquid having a pH of 2.5. That is, in Example 3, a buffer solution was plasma-treated while maintaining the neutrality, and the plasma-treated buffer solution was then acidified by adding a salt.

In Example 4, an aqueous sodium hydroxide solution having a pH of 12 was used as the liquid to be plasma-treated. The first treatment liquid after the plasma treatment had a pH of 7. Sulfuric acid was added to the first treatment liquid to generate a second treatment liquid having a pH of 2.5. That is, in Example 4, an alkaline solution was neutralized by plasma treatment, and the resulting neutral solution was then acidified by adding an acid.

In Example 5, an aqueous sodium hydroxide solution having a pH of 12 was used as the liquid to be plasma-treated. The first treatment liquid after the plasma treatment had a pH of 7. An aqueous 4.5 N sodium hydroxide solution was added to the first treatment liquid to generate a second treatment liquid having a pH of 11.5. That is, in Example 5, an alkaline solution was neutralized by plasma treatment, and the resulting neutral solution was then alkalinized by adding a base.

TABLE 2

| | Comparative | Comparative | Comparative | Comparative | Comparative | Comparative |
|---|---|---|---|---|---|---|
| | Example 1 | Example 2 | Example 3 | Example 4 | Example 5 | Example 6 |
| Material and pH | Standard | Nano-bubble | Nano-bubble | Phosphate | Phosphate | Phosphate |
| | solution | water | water | buffer | buffer | buffer |
| | pH 6 | pH 6 | pH 6 | solution | solution | solution |
| | | | | pH 7.2 | pH 7.2 | pH 7.2 |
| Plasma treatment | Discharge in | — | — | — | — | — |
| | a bubble in | | | | | |
| | liquid | | | | | |
| pH adjustment | — | Addition of | Addition of | Addition of | Addition of | — |
| procedure | | sulfuric acid | NaOH | sulfuric acid | NaOH | |
| pH after | pH 2.4 | pH 3.29 | pH 11.0 | pH 2.5 | pH 11.5 | — |
| treatment/adjustment | | | | | | |

In Comparative Example 1, a standard solution having a pH of 6 was used as the liquid to be plasma-treated. The standard solution was an aqueous sodium sulfate (Na₂SO₄) solution adjusted so as to have a conductivity of 20 mS/m, which was equivalent to that of tap water. Specifically, the standard solution was prepared by diluting 61.3 mg of sodium sulfate with ultra-pure water to 500 mL in a measuring cylinder. In Comparative Example 1, the standard solution was plasma-treated. The treatment liquid after the plasma treatment had a pH of 2.4. In Comparative Example 1, the pH of the plasma-treated liquid was not adjusted.

In Comparative Example 2, nano-bubble water (i.e., plasma-untreated liquid) was prepared. The nano-bubble water was generated by generating nano-bubbles (or ultra fine bubbles) in 4 L of ultra-pure water with a pressurized dissolution ultra fine bubble generator (Ultrafine GALF manufactured by IDEC Corporation). The nano-bubble water had a pH of 6. The nano-bubble water was verified by measurement with a nanoparticle tracking analysis apparatus (Nanosite) manufactured by Quantum Design Japan and was observed to contain 1.6×10⁹ nano-bubbles/mL with a particle size distribution having a peak at 82 nm. In Comparative Example 2, without performing plasma treatment, sulfuric acid was added to the nano-bubble water to prepare a nano-bubble water having a pH of 3.29.

In Comparative Example 3, without performing plasma treatment, an aqueous sodium hydroxide solution was added to the same nano-bubble water (i.e., plasma-untreated liquid) having a pH of 6 as that in Comparative Example 2 to prepare a nano-bubble water having a pH of 11.0.

In Comparative Example 4, a phosphate buffer solution (i.e., plasma-untreated liquid) having a pH of 7.2 was prepared. This phosphate buffer solution was prepared by mixing 302 mg of sodium dihydrogen phosphate dihydrate and 440 mg of disodium hydrogen phosphate and diluting the mixture with ultra-pure water to 500 mL in a measuring cylinder. In Comparative Example 4, without performing plasma treatment, sulfuric acid was added to the phosphate buffer solution to prepare a phosphate buffer solution having a pH of 2.5.

In Comparative Example 5, the same phosphate buffer solution (i.e., plasma-untreated liquid) having a pH of 7.2 as that in Comparative Example 4 was prepared. In Comparative Example 5, without performing plasma treatment, an aqueous sodium hydroxide solution was added to the phosphate buffer solution to prepare a phosphate buffer solution having a pH of 11.5.

In Comparative Example 6, the same phosphate buffer solution (i.e., plasma-untreated liquid) having a pH of 7.2 as that in Comparative Example 4 was prepared. In Comparative Example 6, the phosphate buffer solution was not subjected to plasma treatment and adjustment of pH.

The second treatment liquids of Examples 1 to 5, the plasma-treated liquid of Comparative Example 1, the plasma-untreated liquids having an adjusted pH of Comparative Examples 2 to 5, and the plasma-untreated liquid of Comparative Example 6 were used as the liquid samples for the following decomposition test.

### [3-2. Test of Indigo Carmine Decomposition]

The present inventors performed a test of indigo carmine decomposition for verifying the decomposition ability of each liquid sample. Indigo carmine has a light absorption maximum at a wavelength of 610 nm. That is, when a liquid sample contains indigo carmine, light having the wavelength of 610 nm is strongly absorbed by the indigo carmine. In contrast, if the indigo carmine contained in a liquid sample is decomposed, light having the wavelength of 610 nm is hardly absorbed. Accordingly, the change in absorbance with time, when a liquid sample and indigo carmine are mixed, can be used as an index of the decomposition ability of the liquid sample.

Accordingly, the changes with time in absorbance for light having the wavelength of 610 nm in various liquid samples containing indigo carmine were measured with a spectrometer. The measurement was performed by the following two processes.

In a first measuring process, 11 μL of ultra-pure water containing 2000 ppm of indigo carmine was dropped on a glass cell for spectrophotometer, and 2.2 mL of a liquid sample having a pH adjusted to a desired level was added thereto. Immediately, pipetting was performed to start the measurement of absorbance. That is, the initial concentration of indigo carmine in this measuring process is 10 ppm.

In a second measuring process, the pH is adjusted after the start of absorbance measurement. That is, the measurement of absorbance of the first treatment liquid was started in accordance with the first measuring process, and a pH regulator was then added to the first treatment liquid to generate a second treatment liquid. As a result, the decomposition of indigo carmine by the generated second treatment liquid can be precisely measured. The second measuring process is suitable when the ability of decomposing indigo carmine is high.

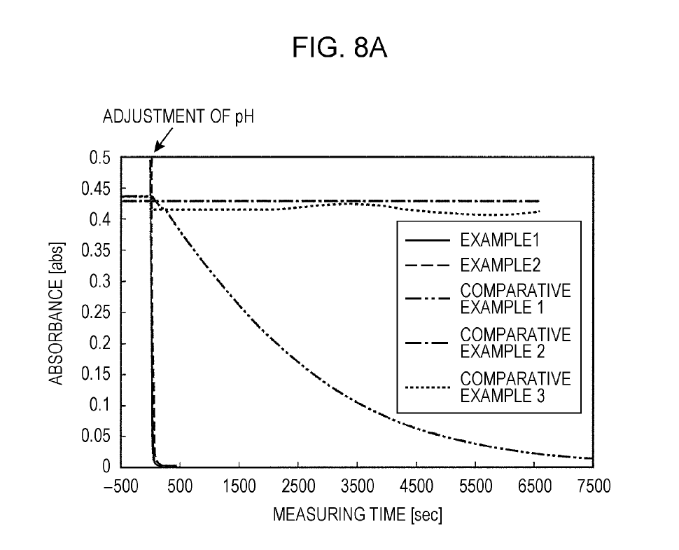

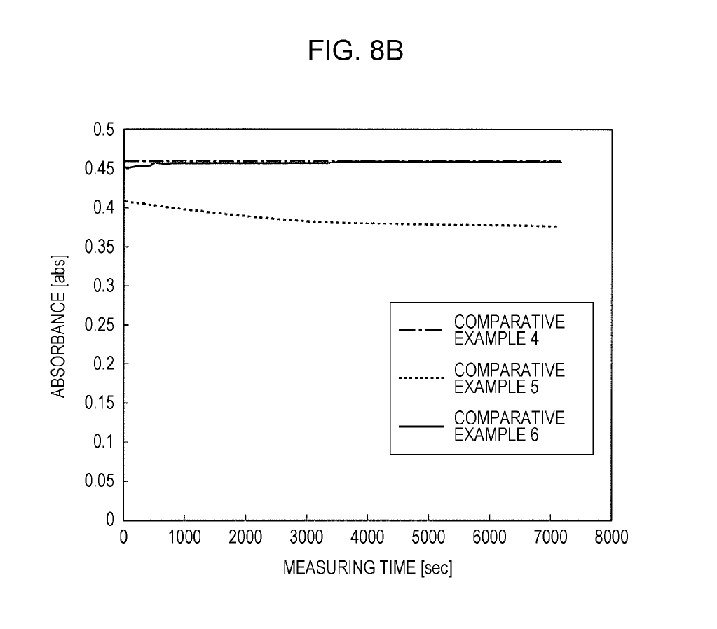

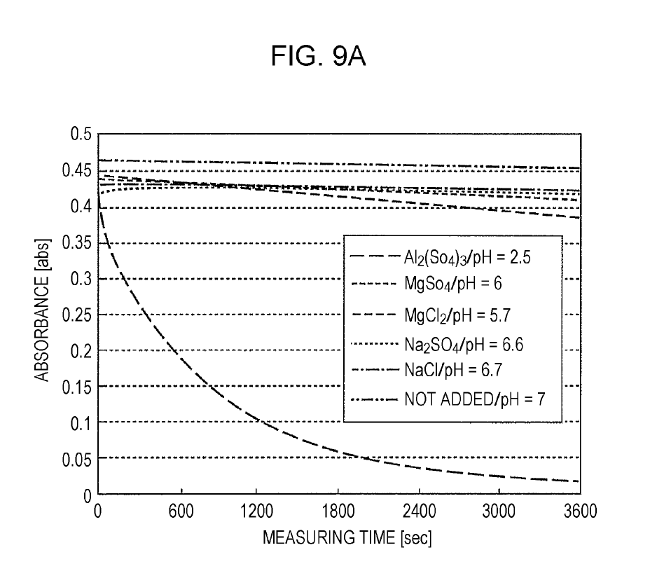

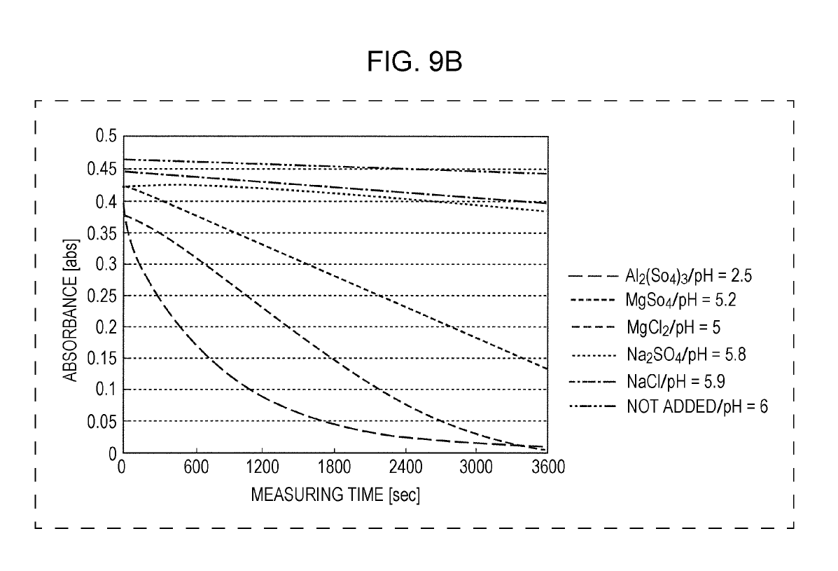

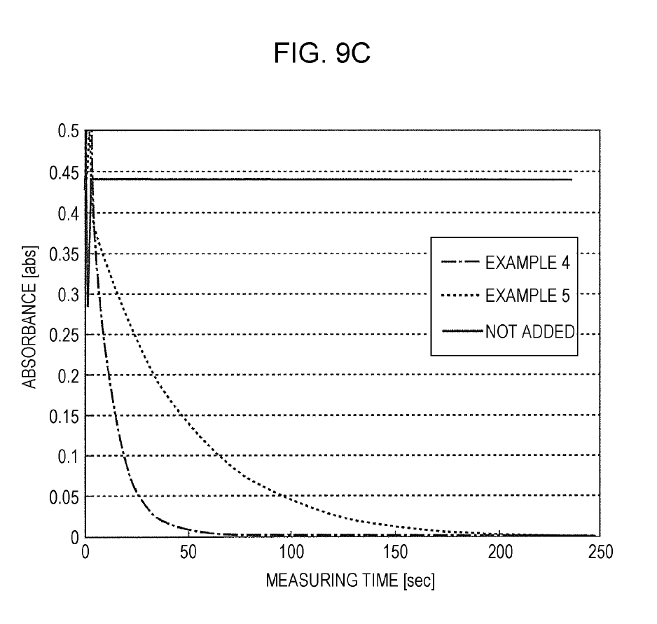

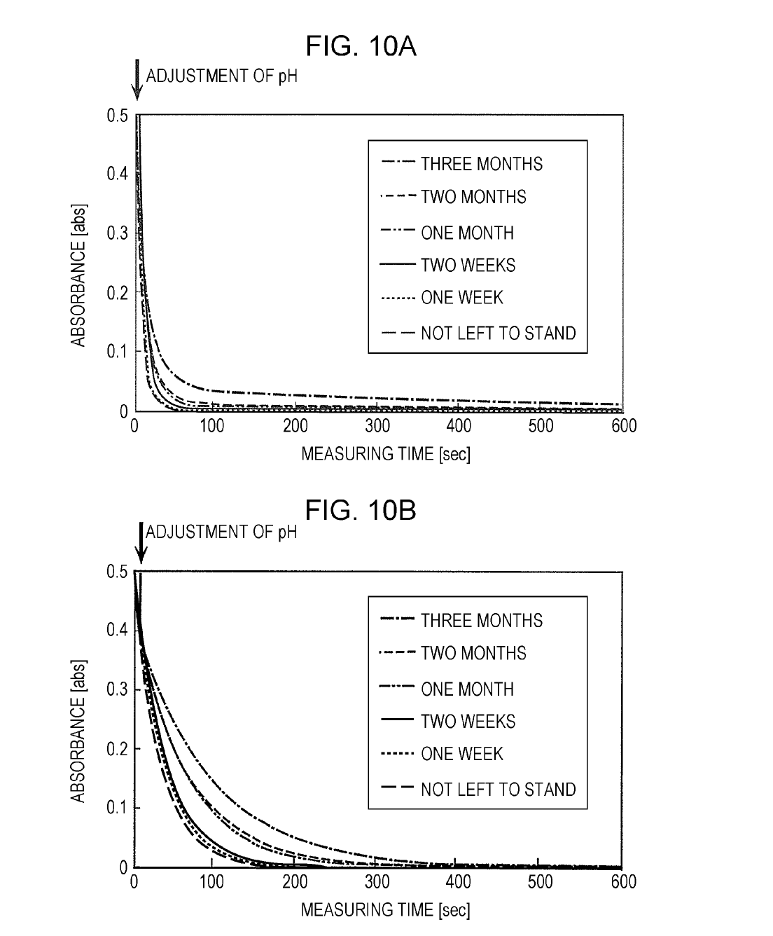

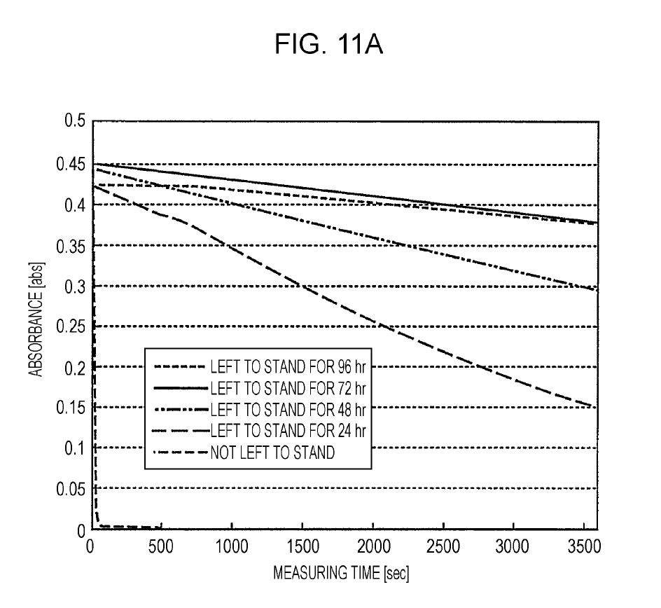

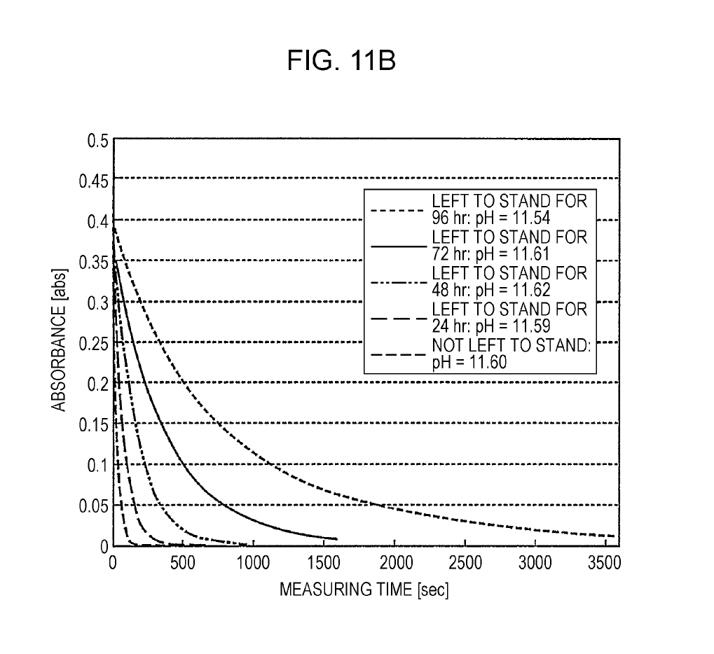

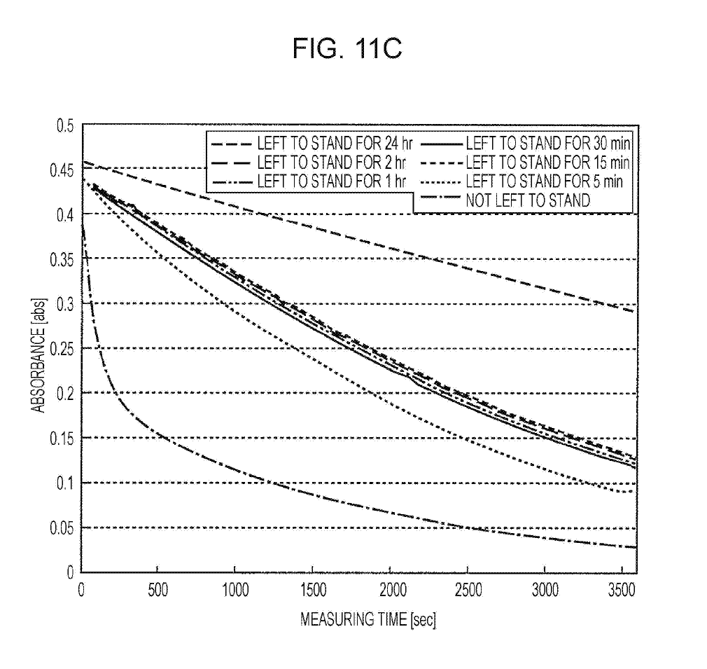

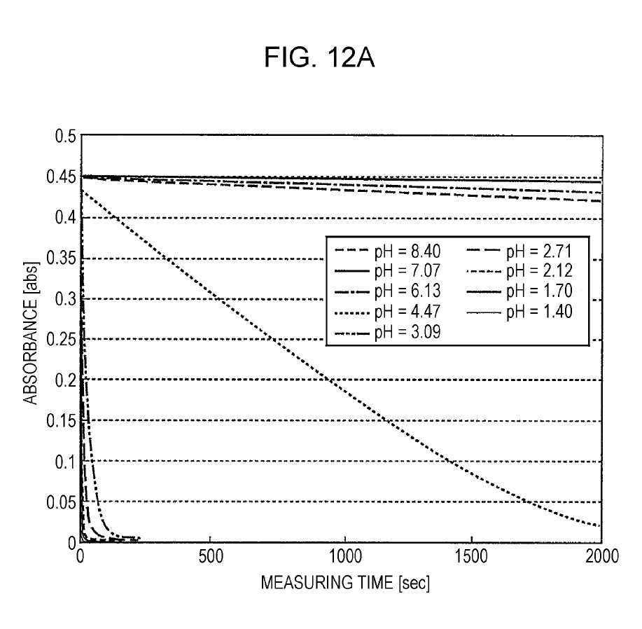

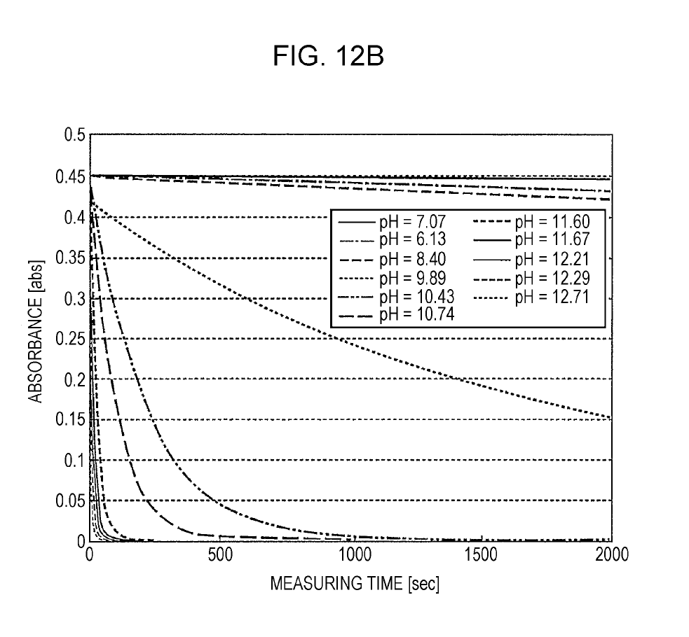

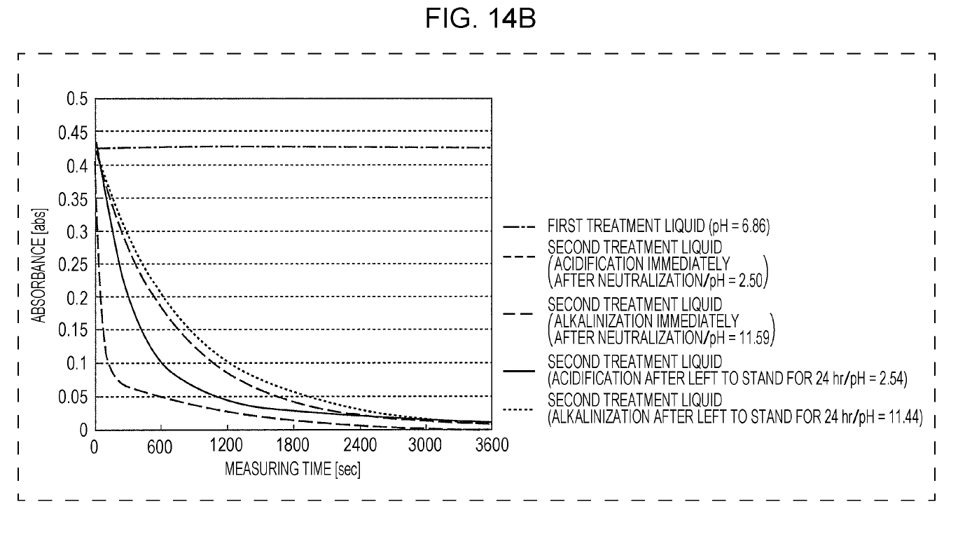

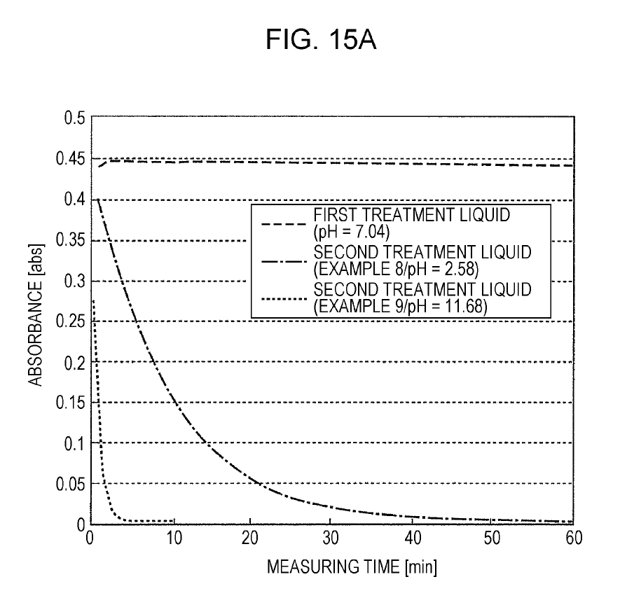

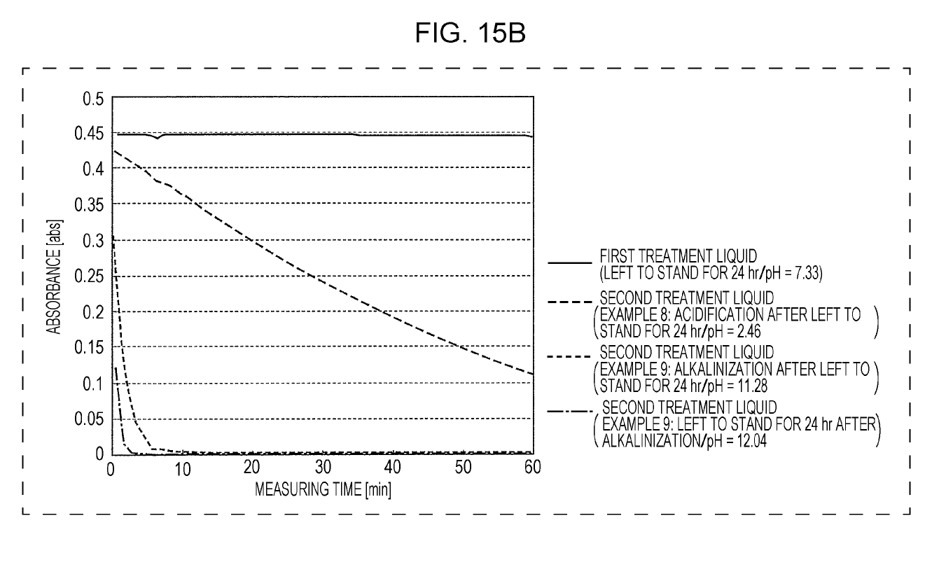

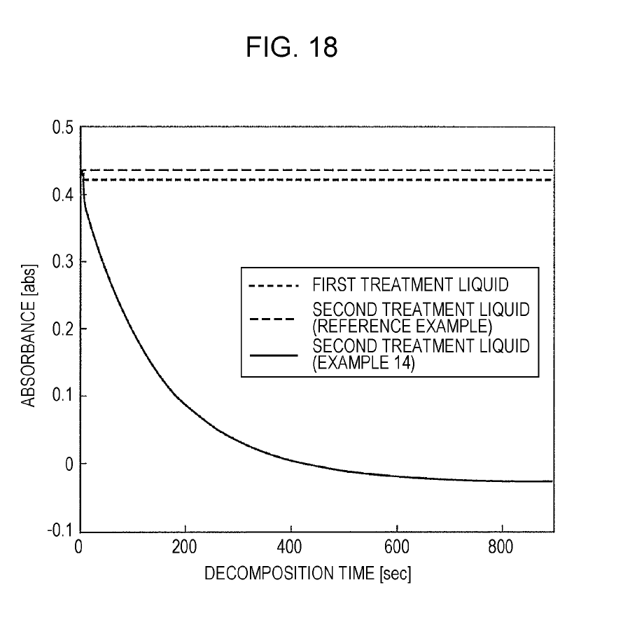

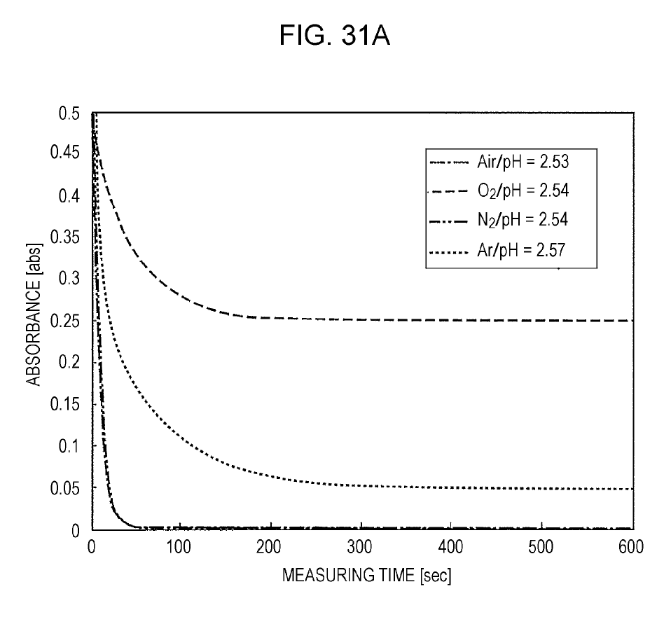

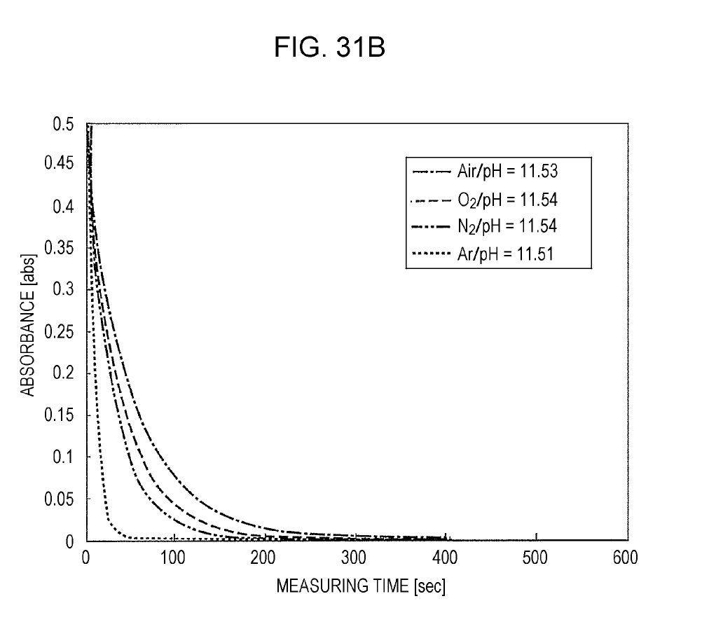

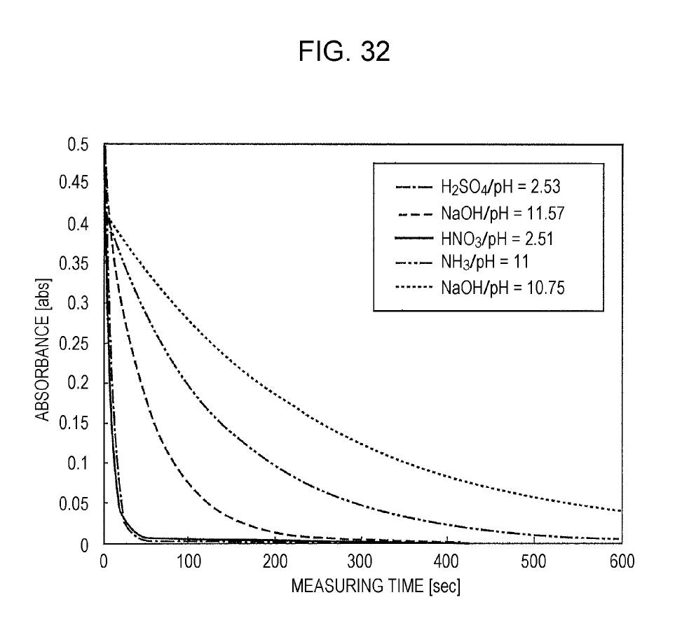

In the experiment data described below, samples measured by the first measuring process are the samples shown in FIG. 8B, the samples not containing Al₂(SO₄)₃ shown in FIGS. 9A and 9B, the samples left to stand after acidification or alkalinization shown in FIGS. 11A and 11B, the samples shown in FIG. 11C, the first treatment liquids shown in FIGS. 14A and 14B, the first treatment liquids shown FIGS. 15A and 15B, and the first treatment liquid shown in FIG. 18. In the experiment data described below, samples measured by the second measuring process are the samples shown in FIG. 8A, the samples containing Al₂(SO₄)₃ shown in FIGS. 9A and 9B, the samples shown in FIG. 9C, the samples shown in FIGS. 10A and 10B, the samples measured immediately after acidification or alkalinization shown in FIGS. 11A and 11B, the samples having a pH of 3.09 or less shown in FIG. 12A, the samples having a pH of 10.43 or more shown in FIG. 12B, the second treatment liquids shown in FIGS. 14A and 14B, the second treatment liquids shown in FIG. 18, the samples shown in FIGS. 31A and 31B, and the samples shown in FIG. 32.

The decomposition ability, storage stability, and durability of each liquid sample will now be described using the drawings.

### [3-3. Decomposition Ability]

FIG. 8A shows the results of a test of indigo carmine decomposition by the liquid samples of Examples 1 and 2 and Comparative Examples 1 to 3. Herein, the absorbance was measured in accordance with the second measuring process. On the horizontal axis in FIG. 8A, the zero point corresponds to the time at which the pH was adjusted, i.e., the time at which a pH regulator was added.

As shown in FIG. 8A, in Comparative Example 1, the absorbance decreased with time. The liquid sample of Comparative Example 1 contained active species produced by plasma treatment, and the active species probably decomposed indigo carmine.

As shown in FIG. 8A, in Examples 1 and 2, the absorbance sharply decreased by the addition of a pH regulator. That is, in Examples 1 and 2, indigo carmine was rapidly decomposed, compared to that in Comparative Example 1.

That is, it was demonstrated that the liquid samples in Examples 1 and 2 had considerably high decomposition ability compared to the liquid sample of Comparative Example 1.

The reason for the above-noted test results is assumed as follows. The treatment liquid generation apparatus 10 generates nano-bubbles and/or micro-bubbles in the liquid 90 by the shock waves due to electric discharge in a bubble 91 in the liquid 90. The micro-bubble is a fine bubble, which has a diameter of 1 μm or more, for example. The nano-bubble is an ultra-fine bubble, which has a diameter of less than 1 μm, for example. The nano-bubbles and/or micro-bubbles contain a gas exposed to plasma 92, and thereby contain active species and/or chemical species, such as ions, molecules, or radicals. These bubbles may cause the active species and/or chemical species to efficiently move into the liquid according to the pH of the liquid. These active species and chemical species may be stably present in the first treatment liquid having a pH of 6 or more and 9 or less, and may be activated by reducing the pH to less than 6 or increasing the pH to higher than 9 of the first treatment liquid to produce other active species, such as radicals, on this occasion. The nano-bubbles and/or micro-bubbles may be stably present in the first treatment liquid having a pH of 6 or more and 9 or less, and may be collapsed by reducing the pH to less than 6 or increasing the pH to higher than 9 of the first treatment liquid to produce other active species, such as radicals, on this occasion. With these assumed reasons, it is assumed that the adjustment of the pH of the liquid samples of Examples 1 and 2 produced the active species instantly within a short period of time and activates the liquid samples to thereby dramatically improve the decomposition ability. It is noted that the analysis of the reaction products demonstrates that the active species prepared by acidification of a plasma-treated liquid and the active species prepared by alkalinization of a plasma-treated liquid are different from each other.

As obvious from FIG. 8A, the liquid samples of Comparative Examples 2 and 3, i.e., the bubble water not subjected to plasma treatment, had significantly low decomposition ability, compared to the liquid samples of Examples 1 and 2, or did not substantially have decomposition ability. This is probably caused by that the liquid samples of Comparative Examples 2 and 3 contain nano-bubbles, but were not plasma-treated. That is, the comparison of Examples 1 and 2 with Comparative Examples 2 and 3 suggests that the inclusion of a gas brought into contact with plasma in nano-bubbles contributes to the decomposition ability.

FIG. 8B shows the results of a test of indigo carmine decomposition by the liquid samples of Comparative Examples 4 to 6.

As obvious from FIG. 8B, the liquid samples of Comparative Examples 4 and 6 did not have decomposition ability. The liquid sample of Comparative Example 5 hardly had decomposition ability. As generally known, indigo carmine partially forms a leuco structure in an alkaline solution having a pH of 11 or more to reduce the absorbance at 610 nm, which is the cause of the low initial absorbance in Comparative Example 5. The reduction in the absorbance is reversible, and the absorbance therefore returns to a value equivalent to that in Comparative Example 4 or 6 by adjusting the pH to 11 or less. However, indigo carmine is gradually decomposed when continuously mixed with an alkaline solution having a pH of 11.5 or more for a long time, resulting a reduction in absorbance.

FIG. 9A shows the results of a test of indigo carmine decomposition by the first treatment liquid and the second treatment liquids of Example 3 and various Reference Examples. The explanatory notes in FIG. 9A show the salts added to the first treatment liquid and the pH of the second treatment liquid.

The salt added to the first treatment liquid was any of sodium chloride (NaCl), sodium sulfate (Na₂SO₄), magnesium sulfate (MgSO₄), magnesium chloride (MgCl₂), and aluminum sulfate (Al₂(SO₄)₃). Among these salts, the treatment liquid containing aluminum sulfate was the liquid sample of Example 3, and treatment liquids containing other salts were the liquid samples of Reference Examples. The liquid sample of Example 3 was an acidified liquid, and the liquid samples of Reference Examples were still neutral liquids excluding the liquid sample containing magnesium chloride and thereby slightly acidified. FIG. 9A also shows, for comparison, the results of the first treatment liquid of Example 3 (“NOT ADDED” in the graph) generated by plasma treatment of a phosphate buffer solution having a pH of 8.5.

As shown in FIG. 9A, in the neutral liquid samples of Reference Examples, the absorbance hardly changed, showing that indigo carmine was hardly decomposed. In contrast, in the liquid samples of Example 3, the absorbance decreased with time, showing decomposition of indigo carmine.

FIG. 9B shows the results of a test of indigo carmine decomposition in other various Examples. Herein, a phosphate buffer solution having a pH of 7.2 was plasma-treated to generate a first treatment liquid having a pH of 6. Subsequently, the salts shown in FIG. 9A were added to the first treatment liquid to generate liquid samples. The various liquid samples subjected to the decomposition test shown in FIG. 9B contained the same salts as those contained in the liquid samples in the decomposition test shown in FIG. 9A, but had different pH levels. The various liquid samples in the decomposition test shown in FIG. 9B were acidified plasma-treated liquids (i.e., second treatment liquids), except the first treatment liquid. As shown in FIG. 9B, in the second treatment liquids containing aluminum sulfate, magnesium chloride, or magnesium sulfate and thereby having a pH of less than 6, indigo carmine was decomposed.

These experimental results demonstrate that the pH regulator is not limited to acids and bases. The pH regulator may be any material that can acidify or alkalinize a first treatment liquid.

FIG. 9C shows the results of a test of indigo carmine decomposition by the first treatment liquids and the second treatment liquids of Examples 4 and 5. In the explanatory notes in FIG. 9C, the first treatment liquids of Examples 4 and 5 are shown as “NOT ADDED”. Examples 4 and 5 were different from Examples 1 to 3 in that the liquid to be plasma-treated was an aqueous sodium hydroxide solution.

As shown in FIG. 9C, in both Examples 4 and 5, the absorbance decreased with time, showing decomposition of indigo carmine. It was accordingly demonstrated that the second treatment liquid had high decomposition ability even if the liquid to be plasma-treated was not a buffer solution. That is, buffering properties are not necessary conditions. However, a buffer solution has a function of stabilizing pH and is therefore easy to handle in generation of a first treatment liquid, adjustment of pH, and storage.

The first treatment liquids (pH: 7) of Examples 4 and 5 hardly decomposed indigo carmine. That is, a first treatment liquid neutralized by plasma treatment does not substantially have decomposition ability if it is not acidified or alkalinized.

### [3-4. Storage Stability]

The storage stability of the potential decomposition ability of a first treatment liquid will be described using FIGS. 10A and 10B.

FIG. 10A shows the results of a test of indigo carmine decomposition by the liquid sample of Example 1. The various liquid samples used in the decomposition test shown in FIG. 10A were prepared by leaving a plasma-treated phosphate buffer solution (i.e., first treatment liquid) to stand for the respective predetermined periods of time at an ordinary temperature (e.g., 20° C.) and then adding sulfuric acid (i.e., pH regulator) to the buffer solution. On the horizontal axis in FIG. 10A, the zero point corresponds to the time at which sulfuric acid was added to the phosphate buffer solution.

As shown in FIG. 10A, the decomposition rates of indigo carmine were substantially the same between the liquid sample not left to stand and the liquid sample left to stand for three months. Accordingly, the potential decomposition ability of the plasma-treated liquid can be preserved in a neutral state for a long period of time. The acidified plasma-treated liquid had high decomposition ability regardless of the storage time.

FIG. 10B shows the results of a test of indigo carmine decomposition by the liquid sample of Example 2. The various liquid samples used in the decomposition test shown in FIG. 10B were prepared by leaving a plasma-treated phosphate buffer solution (i.e., first treatment liquid) to stand for the respective predetermined periods of time at an ordinary temperature and then adding an aqueous sodium hydroxide solution (i.e., pH regulator) to the buffer solution. On the horizontal axis in FIG. 10B, the zero point corresponds to the time at which the aqueous sodium hydroxide solution was added to the phosphate buffer solution.

As shown in FIG. 10B, the decomposition rates of indigo carmine were substantially the same between the liquid sample not left to stand and the liquid sample left to stand for three months. Accordingly, the potential decomposition ability of the plasma-treated liquid can be preserved in a neutral state for a long period of time. The alkalinized plasma-treated liquid had high decomposition ability regardless of the storage time.

### [3-5. Durability]

The durability of the decomposition ability of the second treatment liquid will be described using FIGS. 11A and 11B.

FIG. 11A shows the results of a test of indigo carmine decomposition by the liquid sample (i.e., second treatment liquid) of Example 1 left to stand for various predetermined times after the generation of the liquid sample. The liquid sample used in this test was generated by the same materials and the same process as those of the liquid sample of the above-described Example 1, but had a pH slightly different from that of the liquid sample of the above-described Example 1. However, the liquid sample herein is also called the liquid sample of Example 1, for convenience of explanation.

As shown in FIG. 11A, the time necessary for decomposing indigo carmine with the liquid sample of Example 1 was increased with the period of being left to stand. However, for example, even the liquid sample left to stand for 96 hours decomposed indigo carmine. That is, the second treatment liquid retained its decomposition ability at least for 96 hours after the generation of the second treatment liquid.

FIG. 11B shows the results of a test of indigo carmine decomposition by the liquid sample (i.e., second treatment liquid) of Example 2 left to stand for various predetermined times after the generation. The liquid sample used in this test was generated by the same materials and the same process as those of the liquid sample of the above-described Example 2, but had a pH slightly different from that of the liquid sample of the above-described Example 2. However, the liquid sample herein is also called the liquid sample of Example 2, for convenience of explanation. The actual pH value of the sample left to stand for each period of time is shown in the explanatory notes in FIG. 11B.

As shown in FIG. 11B, the time necessary for decomposing indigo carmine with the liquid sample of Example 2 was increased with the period of time of being left to stand. However, for example, even the liquid sample left to stand for 96 hours decomposed indigo carmine. That is, the second treatment liquid retained its decomposition ability at least for 96 hours after the generation of the second treatment liquid.

The comparison of FIG. 11A and FIG. 11B further demonstrates that the second treatment liquid generated by alkalinization of a first treatment liquid had higher durability of the decomposition ability than that of the second treatment liquid generated by acidification of the first treatment liquid. Accordingly, for example, in a case of decomposing an object for a long time, alkalinization of a first treatment liquid can achieve further effective decomposition.

FIG. 11C shows the results of a test of indigo carmine decomposition by the liquid sample (i.e., first treatment liquid) of Comparative Example 1 left to stand for various predetermined times after the generation.

As shown in FIG. 11C, the liquid sample of Comparative Example 1 left to stand for only 5 minutes needed a longer time for decomposing indigo carmine and decreased the decomposition ability. The decomposition ability of the liquid sample of Comparative Example 1 continued to decrease with the elapse of the time and highly decreased at the elapsed time of 24 hours.

The results described above demonstrate that the liquid samples of Examples 1 and 2 had excellent durability of decomposition ability also in comparison with the liquid sample of Comparative Example 1.

### [3-6. pH and Decomposition Ability]

The relationship between the pH and the decomposition ability of the second treatment liquid will now be described.

FIG. 12A shows the results of a test of indigo carmine decomposition by the liquid samples of various Examples and Reference Examples. The liquid samples used in this test were prepared by generating a first treatment liquid using the same materials and process as those for the liquid sample according to the above-described Example 1 and then adding different types and/or different amounts of pH regulators to the first treatment liquid. The actual pH values of the liquid samples are shown in the explanatory notes in FIG. 12A. Among these liquid samples, the liquid samples having a pH of 1.40 to 4.47 (i.e., acidic liquid samples) correspond to Examples, and the liquid samples having a pH of 6.13 to 8.40 (i.e., neutral liquid samples) correspond to Reference Examples. The liquid samples having a pH of 1.40 to 6.13 were generated by adding sulfuric acid to the first treatment liquid. In the liquid sample having a pH of 7.07, no pH regulator was added to the first treatment liquid. The liquid sample having a pH of 8.40 was generated by adding an aqueous sodium hydroxide solution to the first treatment liquid.

As shown in FIG. 12A, in the liquid samples of Examples, the absorbance of a liquid sample having a lower pH was sharply decreased to show high decomposition ability of the liquid sample. For example, in the liquid samples having a pH of 1.40 to 3.09, the absorbance was decreased to almost zero within 100 seconds. In the liquid sample having a pH of 4.47, the absorbance was decreased to almost zero at about 2000 seconds.

On the other hand, in the liquid samples having a pH of 6.13, 7.07, or 8.40, the absorbance was hardly decreased. That is, the liquid samples of Reference Examples did not substantially have decomposition ability.

FIG. 12B shows the results of a test of indigo carmine decomposition by the liquid samples of various Examples and Reference Examples. The liquid samples used in this test were prepared by generating a first treatment liquid using the same materials and process as those for the liquid sample according to the above-described Example 1 and then adding different types and/or different amounts of pH regulators to the first treatment liquid. The actual pH values of the liquid samples are shown in the explanatory notes in FIG. 12B. Among these liquid samples, the liquid samples having a pH of 6.13 to 8.40 (i.e., neutral liquid samples) correspond to Reference Examples. The liquid samples having a pH of 9.89 to 12.71 (i.e., alkaline liquid samples) correspond to Examples. The liquid sample having a pH of 6.13 was generated by adding sulfuric acid to the first treatment liquid. In the liquid sample having a pH of 7.07, no pH regulator was added to the first treatment liquid. The liquid samples having a pH of 8.40 to 12.71 were generated by adding an aqueous sodium hydroxide solution to the first treatment liquid.

As shown in FIG. 12B, in the liquid samples of Examples, the absorbance of a liquid sample having a higher pH was sharply decreased to show high decomposition ability of the liquid sample. For example, in the liquid samples having a pH of 11.60 to 12.71, the absorbance was decreased to almost zero within 100 seconds. In the liquid samples having a pH of 10.74 or 10.43, the absorbance was decreased to 0.05 or less within 500 seconds. In the liquid sample having a pH of 9.89, the absorbance was about 0.15 at an elapsed time of about 2000 seconds.

On the other hand, in the liquid samples having a pH of 6.13, 7.07, or 8.40, the absorbance was hardly decreased. That is, the liquid samples of Reference Examples did not substantially have decomposition ability. Note that these Reference Examples were the same as those shown in FIG. 12A.

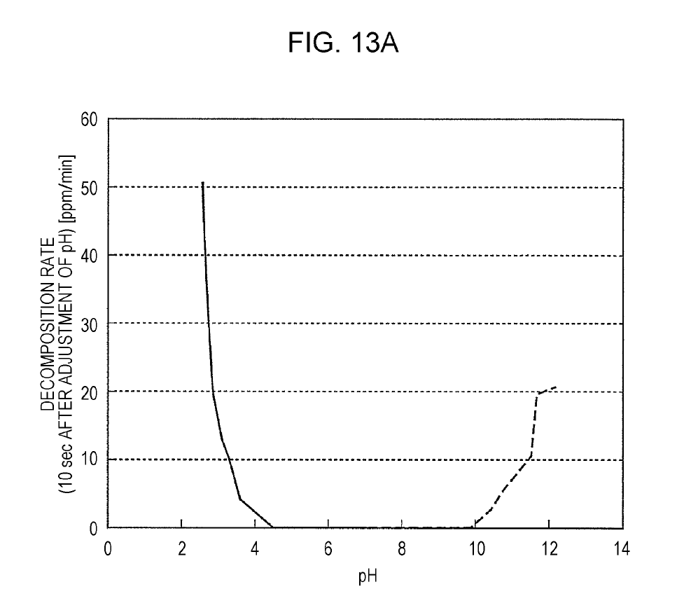

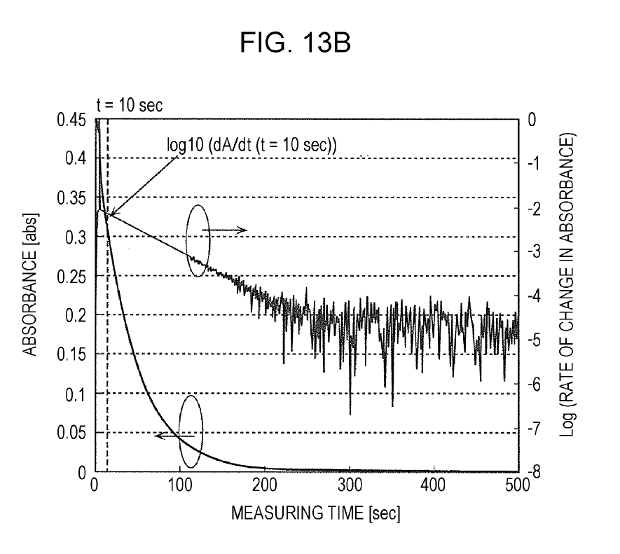

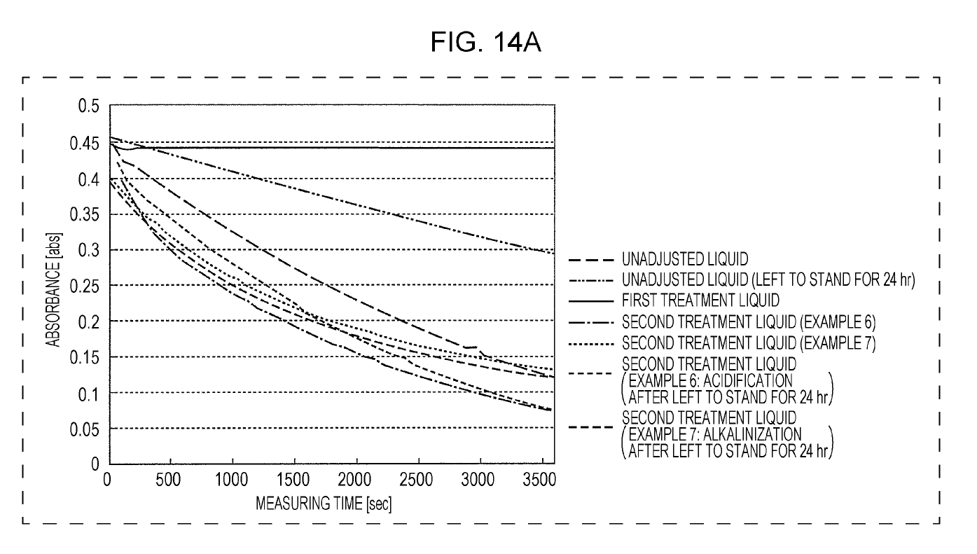

FIG. 13A is a graph summarizing the results shown in FIGS. 12A and 12B by plotting the pH on the horizontal axis and the decomposition rate of indigo carmine on the vertical axis.

The decomposition rate on the vertical axis in FIG. 13A will now be described using FIG. 13B. FIG. 13B is a graph explaining the decomposition rates shown in FIG. 13A.

The decomposition rate of indigo carmine is shown by a change in concentration of indigo carmine with time. Herein, as shown in FIG. 13B, the slope of the absorbance curve (i.e., the rate of change in absorbance) at 10 seconds after the addition of a pH regulator was determined and was multiplied with a prescribed factor (2.72 ppm/abs) to calculate the change in concentration with time (i.e., decomposition rate) of indigo carmine. The predetermined factor was calculated from a calibration curve of the absorbance against the concentration of indigo carmine. The decomposition rates were too high in a pH range of less than 2 or of larger than 12, resulting in impossibility of precise measurement at 10 seconds after the addition of a pH regulator.

As shown in FIG. 13A, the liquid samples having a pH of less than 3.5 or of higher than 10.5 had particularly high decomposition ability.

FIGS. 12A and 12B demonstrate that the liquid samples having a pH of 4.47 or 9.89 also decomposed indigo carmine. Accordingly, a second treatment liquid having a pH of less than 6 or of higher than 9 can be recognized as having high decomposition ability.

### 4. Example (Corresponding to FIG. 6A)

Shown below are the results of a test of indigo carmine decomposition by a neutral first treatment liquid generated by adding a pH regulator after plasma treatment for a predetermined period of time and an acidic or alkaline second treatment liquid generated by adding a pH regulator to the first treatment liquid. Herein, the plasma-treated liquid before the addition of a pH regulator may be referred to as “unadjusted liquid”.

### [4-1. Conditions]

In Examples 6 to 9, the liquid to be plasma-treated contained in the container 20 was plasma-treated with the treatment liquid generation apparatus 10 shown in FIG. 2. The duration of plasma treatment, the applied voltage, and the operations of the circulation pump 80 and gas feeder 56 were the same as those in Examples 1 to 5.

In Examples 6 to 9, the plasma treatment was performed for a predetermined duration (e.g., 30 minutes), instead of plasma treatment for adjusting the pH into a range of 6 or more and 9 or less.

The conditions of each example will now be described in detail using Table 3.

TABLE 3

| | Example 6 | Example 7 | Example 8 | Example 9 |
|---|---|---|---|---|
| Material and pH of liquid | Standard solution | Standard solution | Phosphate buffer | Phosphate buffer |
| to be plasma-treated | pH 6 | pH 6 | solution | solution |
| | | | pH 12 | pH 12 |
| Plasma treatment | Discharge in a | Discharge in a | Discharge in a | Discharge in a |
| | bubble in liquid | bubble in liquid | bubble in liquid | bubble in liquid |
| pH after plasma | pH 2.5 | pH 2.5 | pH 11 | pH 11 |
| treatment | | | | |
| Neutralization procedure | Addition of | Addition of | Addition of sulfuric | Addition of sulfuric |
| | phosphate buffer | phosphate buffer | acid | acid |
| | solution | solution | | |
| pH of first treatment | pH 7 | pH 7 | pH 7 | pH 7 |
| liquid | | | | |
| pH adjustment | Addition of sulfuric | Addition of NaOH | Addition of sulfuric | Addition of NaOH |
| procedure | acid | | acid | |
| pH of second treatment | pH 2.5 | pH 11.5 | pH 2.58 | pH 11.68 |
| liquid | | | | |

In Example 6, a standard solution having a pH of 6 was used as the liquid to be plasma-treated. The unadjusted liquid after plasma treatment had a pH of 2.5. A phosphate buffer solution was added to the unadjusted liquid in an amount of 1 M to generate a first treatment liquid having a pH of 7. Sulfuric acid was added to the first treatment liquid to generate a second treatment liquid having a pH of 2.5. That is, in Example 6, an acidic plasma-treated liquid prepared by plasma treatment of a neutral standard solution was neutralized once and was then acidified by addition of an acid.

In Example 7, a standard solution having a pH of 6 was used as the liquid to be plasma-treated. The unadjusted liquid after plasma treatment had a pH of 2.5. A phosphate buffer solution was added to the unadjusted liquid in an amount of 1 M to generate a first treatment liquid having a pH of 7. An aqueous sodium hydroxide solution was added to the first treatment liquid to generate a second treatment liquid having a pH of 11.5. That is, in Example 7, an acidic plasma-treated liquid prepared by plasma treatment of a neutral standard solution was neutralized once and was then alkalinized by addition of a base.

In Example 8, a phosphate buffer solution having a pH of 12 was used as the liquid to be plasma-treated. The unadjusted liquid after plasma treatment had a pH of 11. Sulfuric acid was added to the unadjusted liquid to generate a first treatment liquid having a pH of 7. Sulfuric acid was further added to the first treatment liquid to generate a second treatment liquid having a pH of 2.58. That is, in Example 8, an alkaline plasma-treated liquid prepared by plasma treatment of an alkaline buffer solution was neutralized once and was then acidified by addition of an acid.

In Example 9, a phosphate buffer solution having a pH of 12 was used as the liquid to be plasma-treated. The unadjusted liquid after plasma treatment had a pH of 11. Sulfuric acid was added to the unadjusted liquid to generate a first treatment liquid having a pH of 7. An aqueous sodium hydroxide solution was added to the first treatment liquid to generate a second treatment liquid having a pH of 11.68. That is, in Example 9, an alkaline plasma-treated liquid prepared by plasma treatment of an alkaline buffer solution was neutralized once and was then alkalinized by addition of a base.

The unadjusted liquids, first treatment liquids, and second treatment liquids of Examples 6 to 9 were used as liquid samples of the decomposition test below.

### [4-2. Test of Indigo Carmine Decomposition]

FIG. 14A shows the results of a test of indigo carmine decomposition by the liquid samples of Examples 6 and 7. FIG. 14A shows the results of decomposition by the unadjusted liquids immediately after plasma treatment, the unadjusted liquids at 24 hours after the plasma treatment, and the first treatment liquids according to Examples 6 and 7; the second treatment liquid acidified immediately after neutralization and the second treatment liquid acidified at 24 hours after the neutralization according to Example 6; and the second treatment liquid alkalinized immediately after neutralization and the second treatment liquid alkalinized at 24 hours after the neutralization according to Example 7.

As shown in FIG. 14A, the absorbance of the first treatment liquid was hardly changed, showing that the indigo carmine was hardly decomposed. The absorbance of the unadjusted liquid immediately after plasma treatment was decreased, showing that the indigo carmine was decomposed. The unadjusted liquid left to stand for 24 hours after the plasma treatment decomposed indigo carmine, but the decomposition rate was significantly low compared to the liquid to be plasma-treated immediately after the plasma treatment.

The absorbance of the second treatment liquid acidified immediately after neutralization was decreased, showing that the indigo carmine was decomposed. Even the second treatment liquid, prepared by leaving a neutral first treatment liquid to stand for 24 hours after neutralization and then acidifying the first treatment liquid, decomposed indigo carmine. Accordingly, it was demonstrated that the potential decomposition ability of the plasma-treated liquid generated from a standard solution can be retained at least for 24 hours in a neutral state.

In the results shown in FIG. 14A, the decomposition rate of indigo carmine by the second treatment liquid acidified at 24 hours after neutralization was higher than that by the first treatment liquid immediately after the neutralization. That is, the decomposition ability of the plasma-treated liquid is increased by neutralizing the liquid once and then acidifying the liquid again.

As shown in FIG. 14A, the second treatment liquid alkalinized immediately after neutralization and the second treatment liquid alkalinized at 24 hours after neutralization showed the same tendencies as those of Example 6.

FIG. 14B shows the results of a test of indigo carmine decomposition by the liquid samples according to another example. In this test, the first treatment liquid was generated using the same materials and process as those in Examples 6 and 7 except that sodium hydroxide was added to an unadjusted liquid instead of the phosphate buffer solution. The explanatory notes in FIG. 14B show the pH of each liquid sample.

As shown in FIG. 14B, even if an aqueous sodium hydroxide solution was used for neutralization, the results were the same as those in Examples 6 and 7. The decomposition ability of the second treatment liquid of the example was higher than those of the second treatment liquids of Examples 6 and 7. However, since the aqueous sodium hydroxide solution does not have buffering properties, adjustment of the pH to a range of 6 or more and 9 or less is not easy.

The above-described results demonstrate that the potential decomposition ability of a plasma-treated liquid acidified by plasma treatment can be retained by neutralizing the plasma-treated liquid. The decomposition ability of this plasma-treated liquid is enhanced by acidification or alkalinization compared to that of the plasma-treated liquid immediately after plasma treatment.

Although the decomposition ability of the treatment liquid being acidic due to plasma treatment highly changes with time, the high decomposition ability can be retained by neutralizing the treatment liquid immediately after plasma treatment.

FIG. 15A shows the results of a test of indigo carmine decomposition by the liquid samples of Examples 8 and 9. FIG. 15A shows the results of the decomposition test by the first treatment liquid of Examples 8 and 9, the second treatment liquid of Example 8, and the second treatment liquid of Example 9. The explanatory notes in FIG. 15A show the pH of each liquid sample.

As shown in FIG. 15A, the absorbance of the first treatment liquid hardly changed, showing that the indigo carmine was hardly decomposed.

The absorbance of the second treatment liquid of Example 8 was decreased, showing that the indigo carmine was decomposed. The absorbance of the second treatment liquid of Example 9 was sharply decreased, showing that the indigo carmine was rapidly decomposed. That is, the decomposition ability of the second treatment liquid of Example 8 was higher than that of the second treatment liquid of Example 9.

FIG. 15B shows the results of a test of indigo carmine decomposition by the liquid samples of Examples 8 and 9. FIG. 15B shows the results of the decomposition test by the first treatment liquids of Examples 8 and 9 at 24 hours after neutralization; the second treatment liquid of Example 8 acidified at 24 hours after neutralization; the second treatment liquid of Example 9 alkalinized at 24 hours after neutralization; and the second treatment liquid of Example 9 at 24 hours after alkalinization. The liquid samples used in this test had pH values slightly different from those of the liquid samples of the above-described Examples 8 and 9. However, the liquid samples herein are also called the liquid samples of Examples 8 and 9, for convenience of explanation. The explanatory notes in FIG. 15B show the pH of each liquid sample.

As shown in FIG. 15B, the absorbance of the first treatment liquid left to stand for 24 hours hardly changed, showing that the indigo carmine was hardly decomposed.

The absorbance of the second treatment liquid acidified after being left to stand for 24 hours was decreased, showing that the indigo carmine was decomposed. The absorbance of the second treatment liquid alkalinized after being left to stand for 24 hours was sharply decreased, showing that the indigo carmine was rapidly decomposed. Comparison of FIGS. 15A and 15B demonstrates that the second treatment liquid alkalinized after being left to stand for 24 hours had a decomposition rate of indigo carmine higher than that of the second treatment liquid alkalinized immediately after neutralization.

The results described above demonstrate that the potential decomposition ability of the plasma-treated liquid alkalinized by plasma treatment can be retained by neutralization of the plasma-treated liquid. This plasma-treated liquid shows high decomposition ability by acidification or alkalinization.

The above-described results demonstrate that the pH of the liquid before the plasma treatment, i.e., the liquid to be plasma-treated, may be any of neutral, acidic, and alkaline.

### 5. Example (Dilution of First Treatment Liquid)

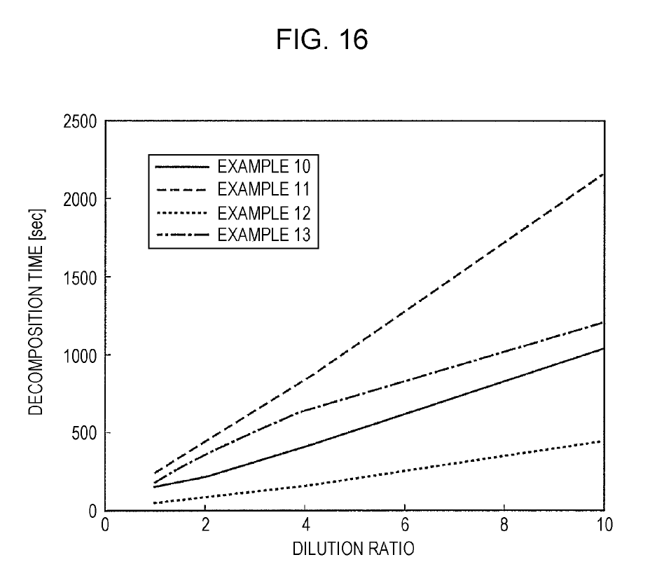

Herein, the results of a test of indigo carmine decomposition by the second treatment liquid generated by acidification or alkalinization of a diluted first treatment liquid will be described using FIG. 16. FIG. 16 shows a relationship between the dilution ratio of each of the first treatment liquids of Examples 10 to 13 and the decomposition time of indigo carmine by each of the second treatment liquids generated from the first treatment liquids.

The liquid samples of Examples 10 to 13 were generated by the following procedure. A 10 mM phosphate buffer solution having a pH of 8.3 was plasma-treated to generate a first treatment liquid having a pH of 7. This first treatment liquid was diluted with a 10 mM phosphate buffer solution having a pH of 7.2 or with ultra-pure water (both were plasma-untreated liquid) to adjust the pH of the first treatment liquid within a range of 6 to 9. Sulfuric acid was added to the diluted first treatment liquid to generate an acidic second treatment liquid. Separately, an aqueous sodium hydroxide solution was added to the diluted first treatment liquid to generate an alkaline second treatment liquid. The liquid sample of Example 10 was generated by diluting the first treatment liquid with a phosphate buffer solution and acidifying the diluted first treatment liquid with sulfuric acid. The liquid sample of Example 11 was generated by diluting the first treatment liquid with a phosphate buffer solution and then alkalinizing the diluted first treatment liquid with an aqueous sodium hydroxide solution. The liquid sample of Example 12 was generated by diluting the first treatment liquid with ultra-pure water and then acidifying the diluted first treatment liquid with sulfuric acid. The liquid sample of Example 13 was generated by diluting the first treatment liquid with ultra-pure water and then alkalinizing the diluted first treatment liquid with an aqueous sodium hydroxide solution.

The test of indigo carmine decomposition by each liquid sample was performed based on the above-described second measuring process. The decomposition time at each dilution ratio was measured based on the results of the decomposition test. The decomposition time herein was determined as the time necessary to change the absorbance by 1×10⁻⁴ abs/sec.

As shown in FIG. 16, in all the second treatment liquids of Examples 10 to 13, the decomposition time was increased with the dilution ratio. That is, the decomposition ability was reduced by dilution, resulting in a reduction in decomposition rate.

Thus, the dilution of the first treatment liquid can adjust the decomposition rate by the second treatment liquid to be subsequently generated and can provide desired decomposition ability. Accordingly, for example, an object can be treated while observing the situation of decomposition. This process can be effectively utilized in, for example, a chemical treatment or a chemical experiment. In addition, the dilution can increase the amount of the second treatment liquid. Accordingly, for example, a large amount of a second treatment liquid can be sprayed in a region having a large area, such as the floor of a bathroom, to allow, for example, effective sterilization.

### Second Embodiment

### 1. Treatment Liquid Generation Apparatus

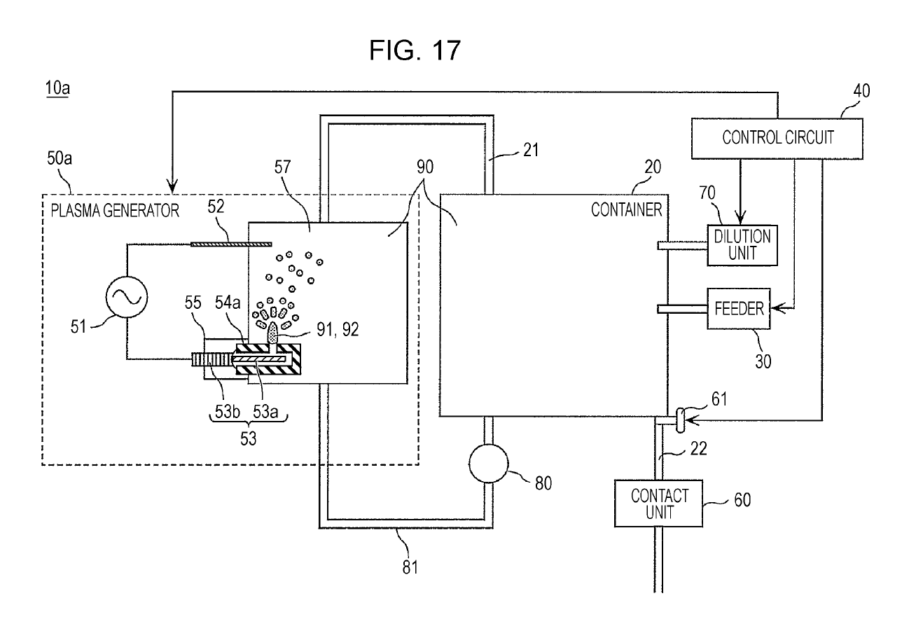

FIG. 17 shows an example of the structure of the treatment liquid generation apparatus 10a according to a Second Embodiment. In this Embodiment, points that are different from the above-described Embodiment will be mainly described.

As shown in FIG. 17, the treatment liquid generation apparatus 10a includes a plasma generator 50a instead of the plasma generator 50 shown in FIG. 2. The plasma generator 50a does not include the gas feeder 56 and includes an insulator 54a instead of the insulator 54.

The insulator 54a is arranged so as to surround the outer surface of the metal electrode portion 53a. The insulator 54a is provided with an opening at a position different from that of the insulator 54 shown in FIG. 2.

When a voltage was applied between the first electrode 52 and the second electrode 53, the second electrode 53 generates heat by the current flowing in the second electrode 53. The generated heat heats the liquid 90 in the circumference of the second electrode 53 and thereby vaporizes the liquid 90 to generate a bubble 91 in the liquid 90. When the generated bubble 91 occludes the opening of the insulator 54a, a voltage is applied to the bubble 91 between the first electrode 52 and the second electrode 53 to cause electric discharge in the bubble 91. As a result, plasma 92 is generate in the bubble 91.

According to the structure described above, the plasma generator 50a can generate plasma 92 in the liquid 90 even when a gas is not supplied to the liquid 90.

### 2. Example (Decomposition Test)

The results of a test of indigo carmine decomposition by the first treatment liquids and the second treatment liquids, which are generated by the treatment liquid generation apparatus 10a, according to the Embodiment will be described using FIG. 18. FIG. 18 shows the results of a test of indigo carmine decomposition by the liquid samples of Example 14 and Reference Example.

Each liquid sample was prepared as follows. A standard solution was plasma-treated by the plasma generator 50a shown in FIG. 17 to generate a first treatment liquid having a pH of 7.3. Sulfuric acid was added to the first treatment liquid to generate a second treatment liquid having a pH of 2.43 as the liquid sample of Reference Example. An aqueous sodium hydroxide solution was added to the first treatment liquid to generate a second treatment liquid having a pH of 11.52 as the liquid sample of Example 14.

As shown in FIG. 18, the absorbance of the first treatment liquid was hardly decreased, showing that the indigo carmine was hardly decomposed. The absorbance of the alkaline second treatment liquid of Example 14 was decreased with time, showing that the indigo carmine was decomposed.

However, the absorbance of the acidic second treatment liquid of Reference Example was hardly decreased even if the time elapsed, showing that the indigo carmine was hardly decomposed. Therefore, if plasma treatment was performed without supplying a gas, the second treatment liquid generated by acidification of a first treatment liquid may hardly have decomposition ability. On the other hand, even if plasma treatment was performed without supplying a gas, the second treatment liquid generated by alkalinization of a first treatment liquid has high decomposition ability.

### 3. Examples (Sterilization Test)

The second treatment liquid can also be used for sterilization.

Table 4 shows the results of a test of sterilization by liquid samples: the first treatment liquids and the second treatment liquids of Examples 15 and 16, the plasma-untreated liquids of Comparative Examples 7 and 8, and the unadjusted liquid of Comparative Example 9.

In Example 15, a phosphate buffer solution was plasma-treated to generate a first treatment liquid, and 2.33 μL of sulfuric acid was added to the first treatment liquid to generate a second treatment liquid. In Example 16, a phosphate buffer solution was plasma-treated to generate a first treatment liquid, and 2.33 μL of an aqueous sodium hydroxide solution was added to the first treatment liquid to generate a second treatment liquid. In Comparative Example 7, 2.33 μL of sulfuric acid was added to a phosphate buffer solution not treated with plasma to generate an acidic phosphate buffer solution. In Comparative Example 8, 2.33 μL of an aqueous sodium hydroxide solution was added to a phosphate buffer solution not treated with plasma to generate an alkaline phosphate buffer solution. In Comparative Example 9, a standard solution was plasma-treated without supplying a gas into the liquid to generate a plasma-treated liquid (i.e., unadjusted liquid). Table 4 shows the conditions for generating each liquid sample.

TABLE 4

| | First | | | | | |
|---|---|---|---|---|---|---|
| | treatment | Second | Second | | | |
| | liquid | treatment | treatment | | | |
| | (Examples 15 | liquid | liquid | Comparative | Comparative | Comparative |
| | and 16) | (Example 15) | (Example 16) | Example 7 | Example 8 | Example 9 |
| Material | Phosphate | Phosphate | Phosphate | Phosphate | Phosphate | Standard |
| | buffer | buffer | buffer | buffer solution | buffer solution | solution |
| | solution | solution | solution | | | |
| Plasma treatment | Discharge in | Discharge in | Discharge in | — | — | Discharge in |
| | a bubble in | a bubble in | a bubble in | | | liquid |
| | liquid | liquid | liquid | | | |
| pH adjustment | — | Sulfuric acid | NaOH | Sulfuric acid | NaOH | — |
| procedure | | | | | | |
| pH after | 7.31 | 2.43 | 11.52 | 2.43 | 11.46 | 7.38 |
| treatment/adjustment | | | | | | |
| Sterilization time | Unsterilized | 54 sec | <10 sec | 309 sec | 298 sec | 120 sec |
| | within 1 hour | | | | | |

The sterilization test will now be described. In the sterilization test, a predetermined amount of E. coli was mixed with each liquid sample to generate a 10⁴ cfu bacterial suspension.

Nine culture samples were prepared by spraying 1 mL of the bacterial suspension onto nine desoxycholate media for each liquid sample with a spiral plater. The reaction of these samples was terminated at 10 seconds, 20 seconds, 30 seconds, 60 seconds, 120 seconds, 300 seconds, 600 seconds, 30 minutes, or 1 hour after the spraying of the bacterial suspension. The reaction was terminated by adding 2.33 μL of a base or acid onto the desoxycholate medium to neutralize the bacterial suspension of an acidic or alkaline liquid sample, or by dropwise adding 50 μL of a 0.1 M sodium thiosulfate solution onto the desoxycholate medium of the liquid sample of Comparative Example 9.

The culture samples were then placed in a thermostat chamber (30° C.), and the bacteria in the culture samples were cultured for 16 hours. The number of bacteria in each culture sample was then counted with a counter.

As shown in Table 4, the sterilization time of the second treatment liquid of Example 16 was less than 10 seconds. The sterilization time is the period from the time at which the liquid is brought into contact with the bacterial suspension until the time at which the viable cell rate is reduced to 1%.

The sterilization time of the second treatment liquid of Example 15 was 54 seconds. The sterilization time of the plasma-treated standard solution of Comparative Example 9 was 120 seconds. Accordingly, the second treatment liquid of Examples 15 and 16 could accomplish the sterilization within a period of time that is a half or shorter than that in the plasma-treated liquid of Comparative Example 9.

The periods of sterilization time of the plasma-untreated liquids of Comparative Examples 7 and 8 were respectively 309 seconds and 298 seconds. Accordingly, the sterilization time of each the second treatment liquids of Examples 15 and 16 was reduced by plasma treatment to one-fifth or less that in each of Comparative Example 7 and 8.

In the first treatment liquids of Examples 15 and 16, the viable cell rate was not reduced to 1% or less even after the elapse of 1 hour, and thus, sterilization was not achieved. That is, the sterilization time can be shortened by acidifying or neutralizing the plasma-treated phosphate buffer solution.

As described above, the second treatment liquid generated by the treatment liquid generation apparatus 10a of the Second Embodiment can be used for sterilization. The results suggest that second treatment liquids generated in other Embodiments have sterilization ability as in the above-described Examples.

### Third Embodiment

### 1. Treatment Liquid Generation Apparatus

In the examples shown in the above-described Embodiments, the second treatment liquid is first generated, and the generated second treatment liquid is then brought into contact with an object, but the procedure is not limited thereto. In a Third Embodiment, the pH of a first treatment liquid is adjusted in a state that the first treatment liquid is in contact with an object. This corresponds to the above-noted second measuring process in the test of indigo carmine decomposition.

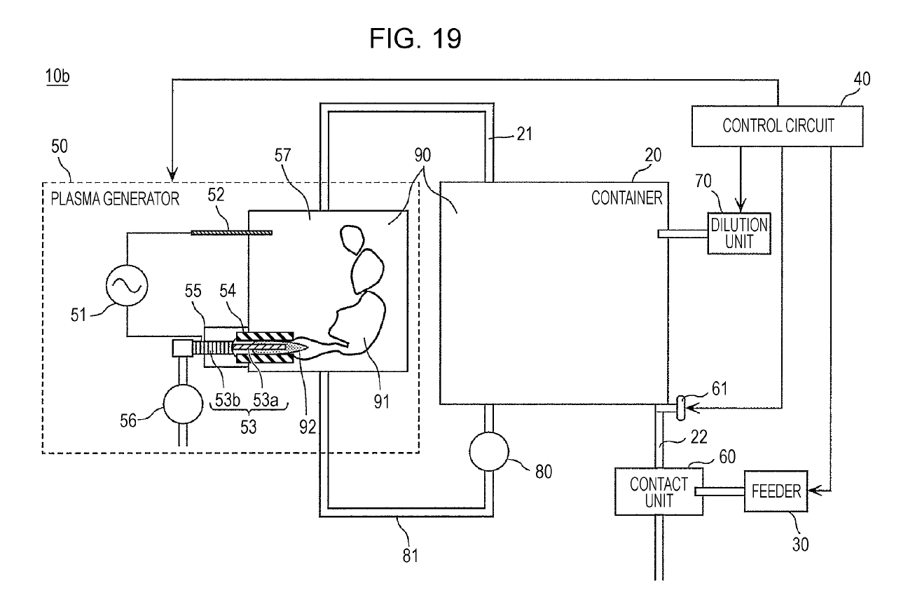

FIG. 19 shows an example of the structure of the treatment liquid generation apparatus 10b according to the Third Embodiment. In this Embodiment, points that are different from the above-described Embodiments will be mainly described.

As shown in FIG. 19, the treatment liquid generation apparatus 10b is different from the treatment liquid generation apparatus 10 shown in FIG. 2 in that the feeder 30 is provided to the contact unit 60 instead of the container 20. Other points are the same as those of the treatment liquid generation apparatus 10 shown in FIG. 2.

According to the structure shown in FIG. 19, the control circuit 40 instructs the feeder 30 to supply a pH regulator to the contact unit 60 to generate a second treatment liquid in a state that a neutral first treatment liquid is in contact with an object. The generated second treatment liquid decomposes and/or sterilizes the object.

As a result, the second treatment liquid and the object can react with each other before the activity of the second treatment liquid is highly decreased. Accordingly, the object can be efficiently decomposed and/or sterilized.

### 2. Operation

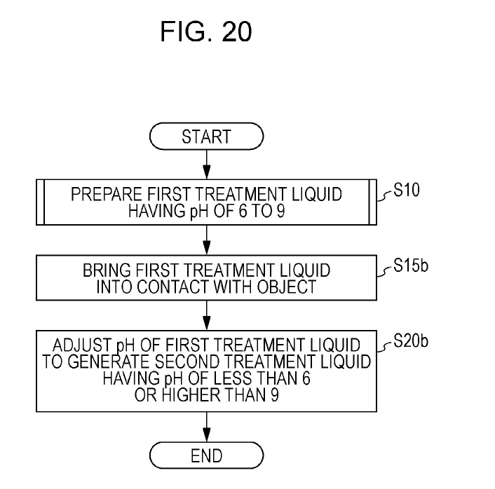

FIG. 20 is a flow chart showing an example of the method of treating an object according to the Third Embodiment.

First, a first treatment liquid having a pH of 6 or more and 9 or less is prepared (S10). The process of the preparation of the first treatment liquid may be the same as that of the above-described Embodiments and are as shown in, for example, FIGS. 5A to 6B.

The treatment liquid generation apparatus 10b then brings the first treatment liquid into contact with an object (S15b). For example, the valve 61 is opened based on the instruction from the control circuit 40 to supply the first treatment liquid from the container 20 to the contact unit 60 through the outlet 22. The contact unit 60 brings the supplied first treatment liquid into contact with the object.

Subsequently, the treatment liquid generation apparatus 10b adjusts the pH of the first treatment liquid being in contact with the object to generate a second treatment liquid having a pH of less than 6 or of higher than 9 (S20b). For example, the feeder 30 supplies a pH regulator to the contact unit 60 based on the instruction from the control circuit 40. The pH regulator is added to the first treatment liquid being in contact with the object and acidifies or alkalinizes the first treatment liquid to generate a second treatment liquid.

The second treatment liquid has high decomposition ability as in the above-described other Embodiments.

Alternatively, when the object itself has a function of acidifying or alkalinizing the plasma-treated liquid, a second treatment liquid may be generated by establishing an environment such that the object can show the acidification or alkalinization function in a state of being in contact with the first treatment liquid. For example, microorganisms that produce, for example, hydrogen sulfide, ammonia, nitrogen oxide, carbon gas, or oxygen are examples of such an object. That is, in such a case, the object also functions as a pH regulator.

As described above, the addition of a pH regulator and the contact of an object and a treatment liquid may be performed in any order.

### Fourth Embodiment

The plasma-treated liquid according to a Fourth Embodiment has the following properties (1) to (3):

(1) when the plasma-treated liquid has a pH of 6 or more and 9 or less, the decomposition rate of indigo carmine is 0.02 ppm/min or less;

(2) when the pH of the plasma-treated liquid is adjusted to 2.5 with a 4.5 N sulfuric acid solution, the decomposition rate of indigo carmine is 0.05 ppm/min or more at 10 seconds after the addition of the sulfuric acid solution; and

(3) when the pH of the plasma-treated liquid is adjusted to 11.5 with an aqueous 4.5 N sodium hydroxide solution, the decomposition rate of indigo carmine is 0.1 ppm/min or more at 10 seconds after the addition of the aqueous sodium hydroxide solution.

These decomposition rates of indigo carmine are calculated by mixing 10 ppm of indigo carmine with a plasma-treated liquid at a temperature of 20° C. and measuring the change in absorbance of light having a wavelength of 610 nm.

The plasma-treated liquid having the above-mentioned properties may be generated by the methods described in the First to Third Embodiments or may be generated by another method. That is, the plasma-treated liquid according to the Fourth Embodiment is not limited by a specific generating apparatus or a specific generating method.

Examples of the plasma-treated liquid according to this Embodiment are shown in Table 5. Table 5 summarizes Examples, Comparative Examples, and Reference Examples described in the First to Third Embodiments.

TABLE 5

| | | Decomposition rate |
|---|---|---|
| Sample | pH | [ppm/min] |
| A | 2.57 | 51.09 |
| B | 2.5 | 23.72 |
| C | 2.5 | 11.64 |
| D | 2.5 | 3.640 |
| E | 11.5 | 14.63 |
| F | 11.5 | 6.913 |
| G | 11.5 | 4.267 |
| H | 11.5 | 2.223 |
| I | 11.5 | 0.267 |
| J | 11.52 | 3.813 |
| K | 6.13 | 0.011 |
| L | 7.07 | 0.001 |
| M | 8.4 | 0.016 |
| N | 2.5 | 2.383 |
| O | 2.5 | 0.212 |
| P | 2.5 | 0.067 |
| Q | 2.5 | <0.0014 |
| R | 7.5 | <0.0014 |
| S | 11.5 | 0.0134 |

Liquid samples A to D were acidic plasma-treated liquids (i.e., second treatment liquids). Liquid sample A was generated by adding sulfuric acid to a plasma-treated phosphate buffer solution. Liquid sample B was generated by diluting a plasma-treated phosphate buffer solution 2 fold with a phosphate buffer solution not treated with plasma and then adding sulfuric acid to the diluted phosphate buffer solution. Liquid sample C was generated by diluting a plasma-treated phosphate buffer solution 4 fold with a phosphate buffer solution not treated with plasma and then adding sulfuric acid to the diluted phosphate buffer solution. Liquid sample D was generated by diluting a plasma-treated phosphate buffer solution 10 fold with a phosphate buffer solution not treated with plasma and then adding sulfuric acid to the diluted phosphate buffer solution.

Liquid samples E to J were alkalinized plasma-treated liquids (i.e., second treatment liquids). Liquid sample E was generated by adding an aqueous sodium hydroxide solution to a plasma-treated phosphate buffer solution. Liquid sample F was generated by diluting a plasma-treated phosphate buffer solution 2 fold with a phosphate buffer solution not treated with plasma and then adding an aqueous sodium hydroxide solution to the diluted phosphate buffer solution. Liquid sample G was generated by diluting a plasma-treated phosphate buffer solution 4 fold with a phosphate buffer solution not treated with plasma and then adding an aqueous sodium hydroxide solution to the diluted phosphate buffer solution. Liquid sample H was generated by diluting a plasma-treated phosphate buffer solution 10 fold with a phosphate buffer solution not treated with plasma and then adding an aqueous sodium hydroxide solution to the diluted phosphate buffer solution. Liquid sample I was generated by neutralizing a plasma-treated standard solution with a buffer component, leaving the resulting plasma-treated liquid (i.e., first treatment liquid) to stand for 24 hours, and then adding an aqueous sodium hydroxide solution to the plasma-treated liquid (i.e., second treatment liquid). Liquid sample J was generated by adding an aqueous sodium hydroxide solution to the plasma-treated standard solution, wherein plasma was generated without supplying a gas into the liquid.

Liquid samples K to M were neutral plasma-treated liquids. Liquid sample K was generated by adding sulfuric acid to a plasma-treated phosphate buffer solution. Liquid sample L was a phosphate buffer solution plasma-treated while maintaining a neutral pH, i.e., a first treatment liquid. Liquid sample M was generated by adding an aqueous sodium hydroxide solution to a plasma-treated phosphate buffer solution.

Liquid samples N to P were plasma-treated standard solutions (i.e., unadjusted liquids). Liquid sample N was a standard solution immediately after plasma treatment. Liquid sample O was a standard solution at 15 minutes after plasma treatment. Liquid sample P was a standard solution at 24 hours after plasma treatment.

Liquid samples Q to S were plasma-untreated liquids. Liquid sample Q was generated by adding sulfuric acid to a phosphate buffer solution not treated with plasma. Liquid sample R was a neutral phosphate buffer solution not treated with plasma. Liquid sample S was generated by adding an aqueous sodium hydroxide solution to a phosphate buffer solution not treated with plasma.

The decomposition rates of indigo carmine by liquid samples A to K, M, and Q to J shown in Table 5 were measured at 10 seconds after the addition of the sulfuric acid or the aqueous sodium hydroxide solution. As shown in Table 5, the second treatment liquids had higher decomposition ability compared to the plasma-untreated liquids. In addition, the second treatment liquids adjusted to be acidic or alkaline had substantially high decomposition ability and excellent durability, compared to unadjusted liquids.

Even if the second treatment liquid was acidified or alkalinized after dilution of the first treatment liquid, the second treatment liquid had high decomposition ability. Furthermore, the generation of a first treatment liquid by plasma may be performed by any process, and the plasma may be generated by supplying a gas or not supplying any gas.

### Fifth Embodiment

### 1. Object Treatment Apparatus

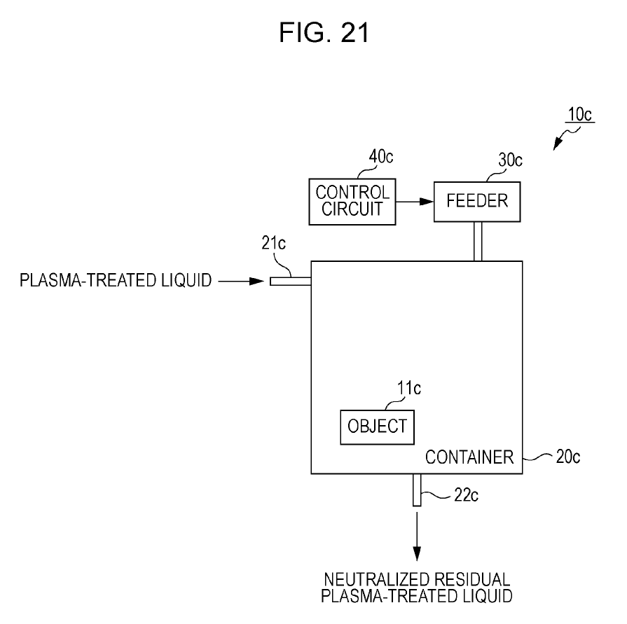

The outline of the object treatment apparatus according to a Fifth Embodiment will be described referring to FIG. 21. FIG. 21 shows the structure of the object treatment apparatus 10c according to the Fifth Embodiment.

The object treatment apparatus 10c allows a plasma-treated liquid to act on an object 11 and then adjusts the pH of the remaining liquid to 6 or more and 9 or less. As shown in FIG. 21, the object treatment apparatus 10c includes a container 20c, a feeder 30c, and a control circuit 40c. The container 20c is provided with an inlet 21c and an outlet 22c. The outlet 22c is for discharging the remained plasma-treated liquid (i.e., residual liquid).

The container 20c in FIG. 21, for example, corresponds to the contact unit 60 in FIG. 2. The container 20c may be formed of the same material as that of the container 20 described in the First Embodiment. The inlet 21c in FIG. 21 is, for example, connected to the outlet 22 in FIG. 2 through a pipe. Accordingly, the plasma-treated liquid flowing into the container 20c from the inlet 21c in FIG. 21 is, for example, the second treatment liquid described in any of the First to Fourth Embodiments. The control circuit 40c in FIG. 21 may be, for example, commonized with the control circuit 40 in FIG. 2. The feeder 30c in FIG. 21 may be, for example, commonized with the feeder 30 in FIG. 2.

In the Fifth Embodiment, the object 11c is contained in the container 20c, and the plasma-treated liquid and the object 11c are brought into contact with each other in the container 20c. As a result, the container 20c contains the residual liquid of the plasma-treated liquid acted on the object 11c.

The feeder 30c supplies a predetermined amount of a pH regulator to the container 20c based on the instruction from the control circuit 40c to adjust the pH of the residual liquid to 6 or more and 9 or less.

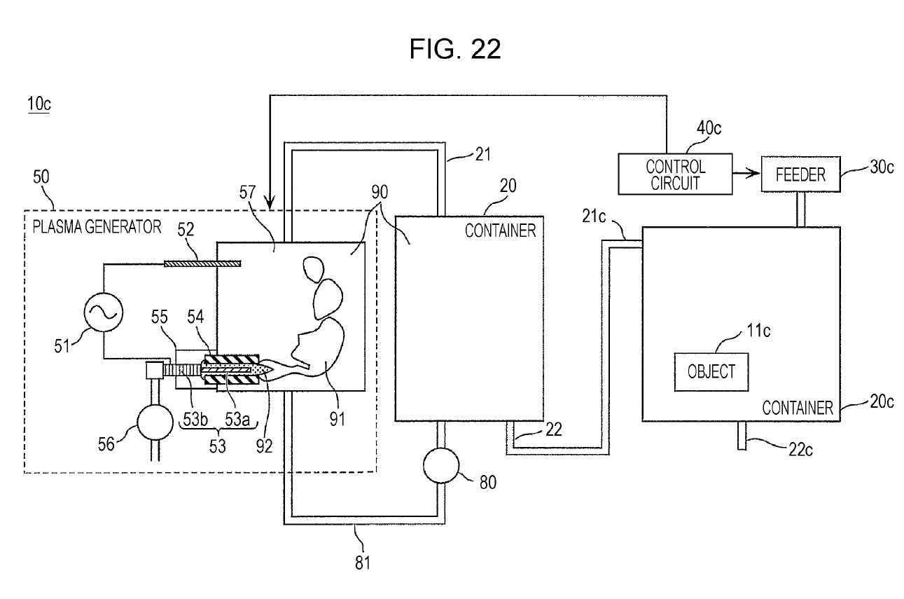

FIG. 22 shows an example of the structure of the object treatment apparatus 10c. Among the components shown in FIG. 22, those having the same reference numbers as the components shown in FIG. 2 can have, for example, the same structures as those described in the First Embodiment.

### 2. Operation

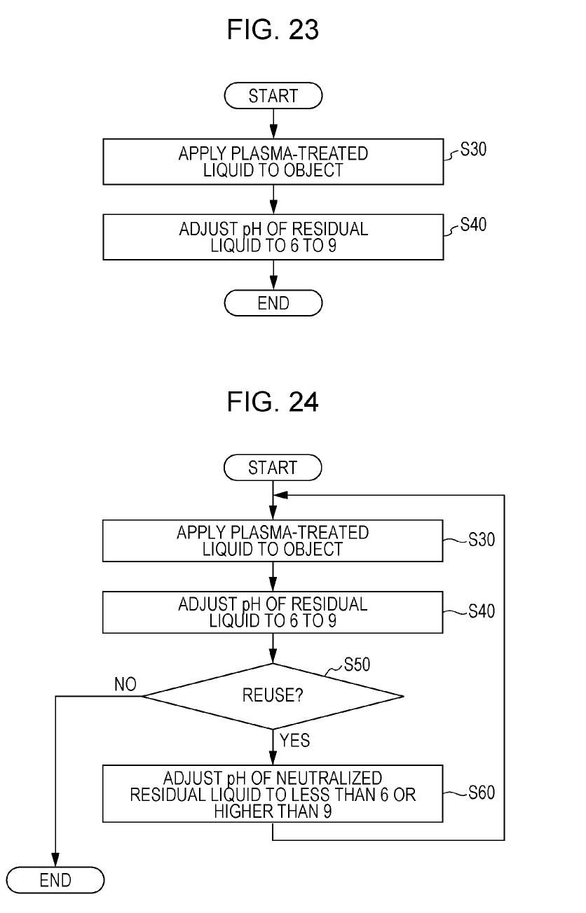

FIG. 23 is a flow chart showing an example of the method of treating an object according to the Fifth Embodiment.

The object treatment apparatus 10c applies a plasma-treated liquid to an object 11c (S10). The plasma-treated liquid is, for example, the second treatment liquid described in the First Embodiment.

Subsequently, the object treatment apparatus 10c adjusts the pH of the liquid remaining in the container 20c to 6 or more and 9 or less (S20). For example, the feeder 30c supplies a pH regulator to the container 20c based on the instruction from the control circuit 40c. For example, the feeder 30c adds a solution containing an acid, base, or salt to the residual liquid.

As a result, the activity of the residual liquid is suppressed, and the residual liquid can be safely discarded.

FIG. 24 is a flow chart showing another example of the method of treating an object according to the Fifth Embodiment. Steps S30 and S40 in FIG. 24 are respectively the same as steps S30 and S40 in FIG. 23, and the descriptions thereof are omitted.

The pH of the residual liquid is adjusted to 6 or more and 9 or less (S40), and the residual liquid is then determined whether it is reused or not (S50). When the residual liquid is reused (the case of “Yes” in S S50), the object treatment apparatus 10c adjusts the pH of the neutralized residual liquid to less than 6 or to higher than 10 (S60). For example, the feeder 30c supplies a pH regulator to the container 20c based on the instruction from the control circuit 40c. For example, the feeder 30c adds a solution containing an acid, base, or salt to the residual liquid. The supplied pH regulator may be the same as or different from the pH regulator supplied in step S40. After step S60, for example, the procedure returns to step S30.

The residual liquid neutralized once is acidified or alkalinized to recover the activity as a plasma-treated liquid. As a result, the plasma-treated liquid can be applied to the object 11c again.

When the residual liquid is not reused (the case of “No” in S30), the procedure ends as it is. In such a case, for example, the residual liquid can be safely discarded.

### 3. Examples

The results of a test of indigo carmine decomposition by plasma-treated liquids will now be described. Specifically, termination of a decomposition reaction by neutralization of a plasma-treated liquid and the subsequent restart of the decomposition reaction by acidification or alkalinization of the plasma-treated liquid will be described.

### [3-1. Conditions]

Table 6 summarizes the conditions of Examples 17 to 19 and Reference Example.

TABLE 6

| | Example 17 | Example 18 | Example 19 | Reference Example |
|---|---|---|---|---|
| Material and pH of liquid | Phosphate buffer | Phosphate buffer | Phosphate buffer | Standard solution |
| to be plasma-treated | solution | solution | solution | pH 6 |
| | pH 8.3 | pH 8.3 | pH 12 | |
| Plasma treatment | Discharge in a | Discharge in a | Discharge in a | Discharge in a |
| | bubble in liquid | bubble in liquid | bubble in liquid | bubble in liquid |
| pH after plasma | pH 6.9 | pH 6.9 | pH 11 | pH 2.4 |
| treatment | | | | |
| Neutralization procedure | — | — | Addition of sulfuric | — |
| | | | acid | |
| pH of first treatment | pH 6.9 | pH 6.9 | pH 7.3 | — |
| liquid | | | | |
| Activation procedure | Addition of sulfuric | Addition of NaOH | Addition of sulfuric | — |
| | acid | | acid | |
| pH of second treatment | pH 2.5 | pH 11.5 | pH 2 .6 | — |
| liquid | | | | |
| Neutralization procedure | Addition of NaOH | Addition of sulfuric | Addition of NaOH | Addition of |
| | | acid | | phosphate buffer |
| | | | | solution |
| pH after the | pH 7 | pH 7 | pH 7.1 | pH 6 |
| neutralization | | | | |
| Oxidation procedure | Addition of sulfuric | Addition of sulfuric | Addition of sulfuric | Addition of sulfuric |
| | acid | acid | acid | acid |
| Alkalinization procedure | Addition of NaOH | Addition of NaOH | Addition of NaOH | Addition of NaOH |
| Object | Indigo carmine | Indigo carmine | Indigo carmine | Indigo carmine |

The liquid samples according to Examples 17 to 19 were produced by the same processes as those in the liquid samples according to Examples 1, 2, and 8, respectively. However, the pH values of the first treatment liquids of Examples 17 to 19 were slightly different from those of the first treatment liquids of Examples 1, 2, and 8, and the pH values of the second treatment liquids of Examples 17 to 19 were slightly different from those of the second treatment liquids of Examples 1, 2, and 8.

The liquid sample according to Reference Example was produced by the same process as that in the liquid sample according to Comparative Example 1. However, the pH value of the first treatment liquid of Reference Example was slightly different from that in Comparative Example 1.

The decomposition test described below was carried out in accordance with the above-described second measuring process. Neutral first treatment liquids according to Examples 17 to 19 and an acidic plasma-treated liquid (i.e., unadjusted liquid) according to Reference Example were prepared as liquid samples. The liquid samples and indigo carmine were mixed, and measurement of the absorbance of this mixture for light having a wavelength of 610 nm was started. The change in absorbance of this mixture was observed while adding sulfuric acid or an aqueous sodium hydroxide solution or a phosphate buffer solution to the mixture at predetermined timing.

### [3-2. Termination and Restart of Decomposition]

### [3-2-1. Plasma-Treated Phosphate Buffer Solution]

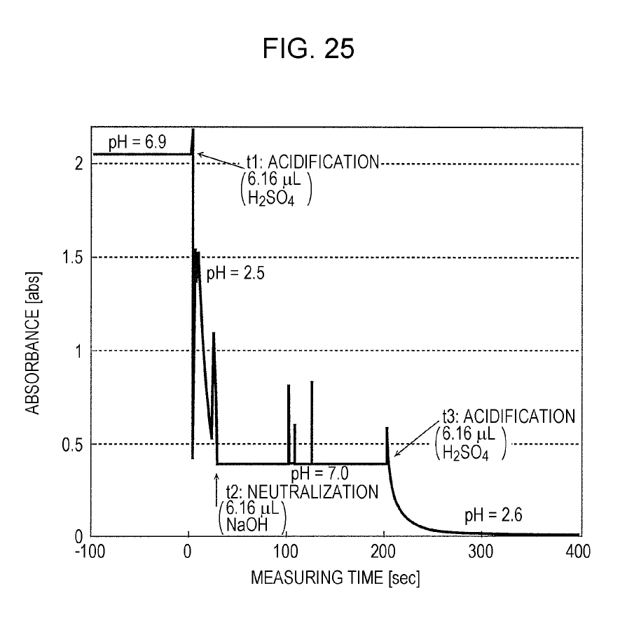

The results of a test of indigo carmine decomposition by the liquid samples according to Examples 17 and 18 will be described referring to FIGS. 25 to 28. On the horizontal axis in FIGS. 25 to 28, the zero point corresponds to the time at which the pH of the neutral phosphate buffer solution (i.e., first treatment liquid) was firstly changed. In FIGS. 25 to 28, t1 shows the time of the first addition of a pH regulator, t2 shows the time of the second addition of a pH regulator, and t3 shows the time of the third addition of a pH regulator. In FIGS. 25 to 28, the liquid sample according to Example 17 refers to the liquid sample prepared by the first addition of sulfuric acid to a neutral liquid. The liquid sample according to Example 18 refers to the liquid sample prepared by first addition of an aqueous sodium hydroxide solution to a neutral liquid.

FIG. 25 shows the results of a first decomposition test by the liquid sample according to Example 17.

Before time t1, the phosphate buffer solution had a pH of 6.9, and the absorbance was not substantially changed.

Sulfuric acid (6.25 μL) was added to the phosphate buffer solution (2.2 mL) at time t1 to change the pH to 2.5. As a result, the absorbance was sharply decreased to show decomposition of indigo carmine, and a residual liquid remained.

An aqueous sodium hydroxide solution (6.16 μL) was added to the phosphate buffer solution (i.e., residual liquid) at time t2 to change the pH to 7.0. As a result, the change in absorbance was substantially stopped to show termination of the decomposition of indigo carmine.

Sulfuric acid (6.16 μL) was added to the phosphate buffer solution (i.e., residual liquid) at time t3 to change the pH to 2.6. As a result, the absorbance was further decreased to show decomposition of indigo carmine.

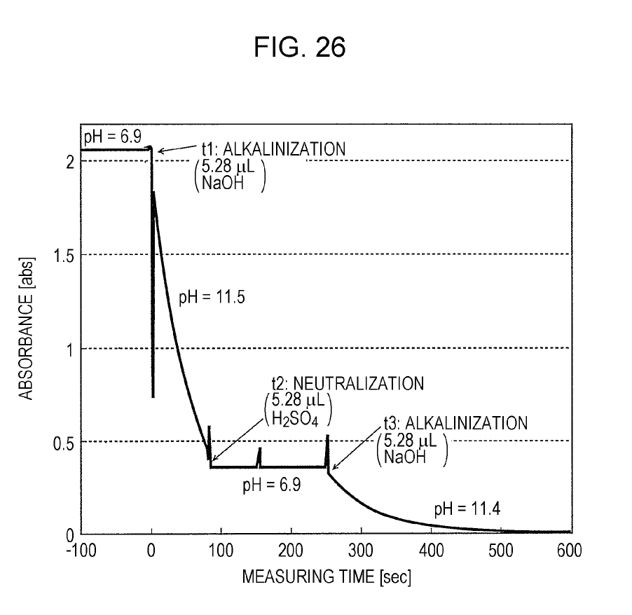

FIG. 26 shows the results of a first decomposition test by the liquid sample according to Example 18.

Before time t1, the phosphate buffer solution had a pH of 6.9, and the absorbance was not substantially changed.

An aqueous sodium hydroxide solution (5.28 μL) was added to the phosphate buffer solution (2.2 mL) at time t1 to change the pH to 11.5. As a result, the absorbance was sharply decreased to show decomposition of indigo carmine, and a residual liquid remained.

Sulfuric acid (5.28 μL) was added to the phosphate buffer solution (i.e., residual liquid) at time t2 to change the pH to 6.9. As a result, the change in absorbance was substantially stopped to show termination of the decomposition of indigo carmine.

An aqueous sodium hydroxide solution (5.28 μL) was added to the phosphate buffer solution (i.e., residual liquid) at time t3 to change the pH to 11.4. As a result, the absorbance was further decreased to show decomposition of indigo carmine.

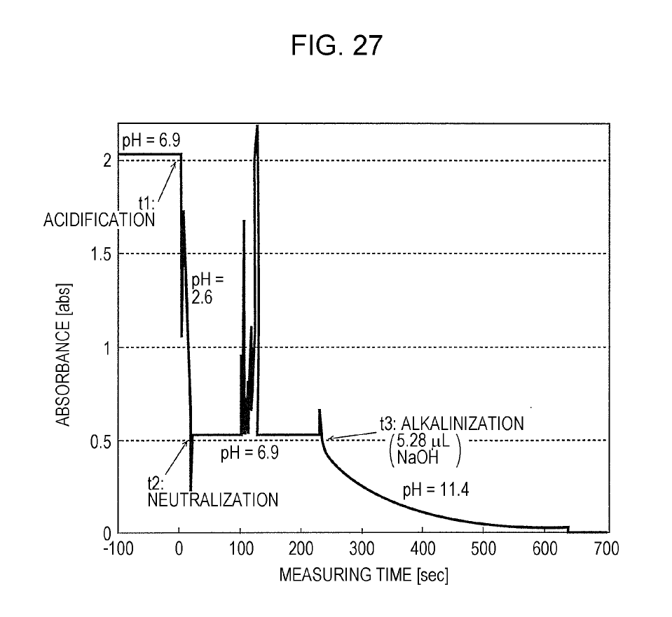

FIG. 27 shows the results of a second decomposition test by the liquid sample according to Example 17.

The same process as that in the measurement described referring to FIG. 25 was carried out until time t2.

An aqueous sodium hydroxide solution (6.16 μL) was added to a phosphate buffer solution (2.2 mL) at time t2 to change the pH to 6.9.

An aqueous sodium hydroxide solution (5.28 μL) was further added to the phosphate buffer solution (i.e., residual liquid) at time t3 to change the pH to 11.4. As a result, the absorbance was further decreased to show decomposition of indigo carmine.

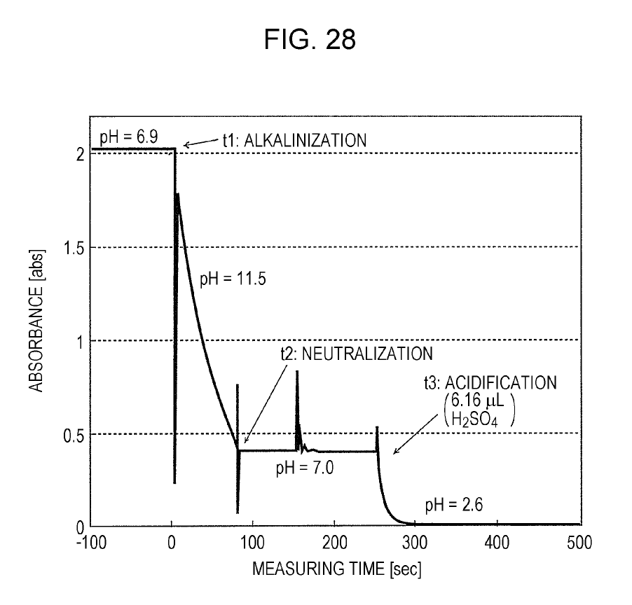

FIG. 28 shows the results of a second decomposition test by the liquid sample according to Example 18.

The same process as that in the measurement described referring to FIG. 26 was carried out until time t2.

Sulfuric acid (5.28 μL) was added to a phosphate buffer solution (2.2 mL) at time t2 to change the pH to 6.9.

Sulfuric acid (6.16 μL) was further added to the phosphate buffer solution (i.e., residual liquid) at time t3 to change the pH to 2.6. As a result, the absorbance was further decreased to show decomposition of indigo carmine.

In FIGS. 25 to 28, the spike-like change in absorbance at each time of t1, t2, and t3 is caused by pipetting for uniformly mixing a phosphate buffer solution and a pH regulator. The spike-like change in absorbance observed between time t2 and time t3 is caused by insertion of an electrode for measuring the pH of the phosphate buffer solution.

As described above, the activity of a plasma-treated liquid is terminated by neutralization and is reactivated by acidification or alkalinization. Accordingly, the activity of a plasma-treated liquid can be controlled by controlling the pH of the plasma-treated liquid. The pH value before the termination of decomposition and the pH value after the restart of decomposition may be the same or different.

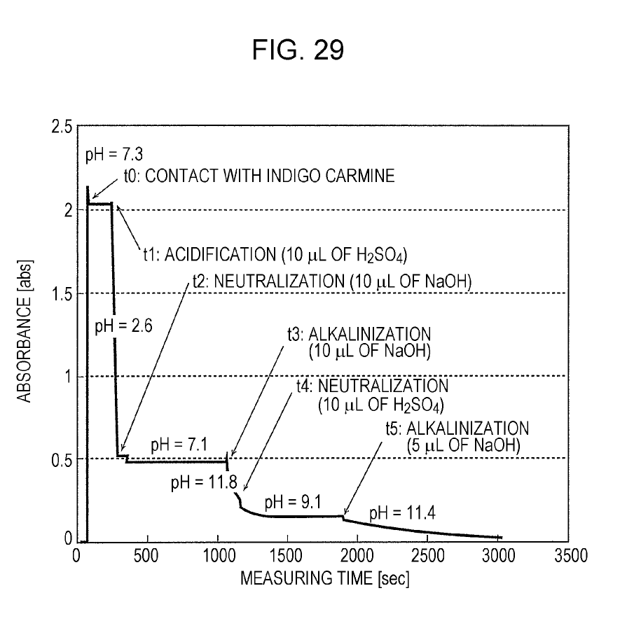

The termination and restart of decomposition may be repeated multiple times. FIG. 29 shows the results of a test of indigo carmine decomposition by the liquid sample of Example 19. In FIG. 29, time t0 shows the time at which a phosphate buffer solution is brought into contact with indigo carmine, and times t1 to t5 show the times for sequentially adding pH regulators after the contact.

A phosphate buffer solution (i.e., first treatment liquid) having a pH of 7.3 was brought into contact with indigo carmine at time t0. However, the absorbance was not substantially changed to show no decomposition of indigo carmine.

Sulfuric acid (10 μL) was added to the phosphate buffer solution (2.5 mL) at time t1 to change the pH to 2.6. As a result, the absorbance was sharply decreased to show decomposition of indigo carmine.

An aqueous sodium hydroxide solution (10 μL) was added to the phosphate buffer solution (i.e., residual liquid) at time t2 to change the pH to 7.1. As a result, the change in absorbance was substantially stopped to show termination of the decomposition of indigo carmine.

An aqueous sodium hydroxide solution (10 μL) was further added to the phosphate buffer solution (i.e., residual liquid) at time t3 to change the pH to 11.8. As a result, the absorbance was further decreased to show decomposition of indigo carmine.

Sulfuric acid (10 μL) was added to the phosphate buffer solution (i.e., residual liquid) at time t4 to change the pH to 9.1. As a result, the change in absorbance was substantially stopped to show re-termination of the decomposition of indigo carmine.

An aqueous sodium hydroxide solution (5 μL) was added to the phosphate buffer solution (i.e., residual liquid) at time t5 to change the pH to 11.4. As a result, the absorbance was further decreased to show decomposition of indigo carmine.

### [3-2-2. Plasma-Treated Standard Solution]

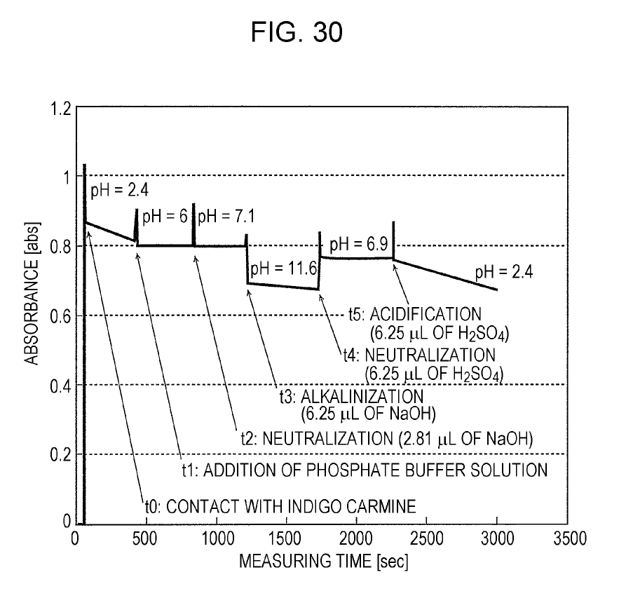

The results of a test of indigo carmine decomposition by the liquid sample according to Reference Example will be described referring to FIG. 30. In FIG. 30, time t0 shows the time at which indigo carmine is mixed with an acidic standard solution (i.e., plasma-treated liquid), time t1 shows the time at which a phosphate buffer solution is added to a standard solution, and times t2 to t5 show the times for sequentially adding pH regulators to the standard solution.

A standard solution (2.5 mL) having a pH of 2.4 was brought into contact with indigo carmine at time t0. As a result, the absorbance was gradually decreased.

A phosphate buffer solution (concentration: 1 M, 25 μL) was added to the standard solution (i.e., residual liquid) at time t1 to change the pH to 6. As a result, the change in absorbance was substantially stopped to show termination of the decomposition of indigo carmine.

An aqueous sodium hydroxide solution (2.81 μL) was added to the standard solution (i.e., residual liquid) at time t2 to change the pH to 7.1. In also this step, the absorbance was not substantially changed to show no decomposition of indigo carmine.

An aqueous sodium hydroxide solution (6.25 μL) was added to the standard solution (i.e., residual liquid) at time t3 to change the pH to 11.6. As a result, the absorbance was sharply decreased and then gradually decreased. The sharp decrease of the absorbance was caused by that a part of the indigo carmine formed a leuco structure in the strong alkaline solution. Accordingly, the gradual decrease in absorption between time t3 and time t4 corresponds to decomposition of indigo carmine.

Sulfuric acid (6.25 μL) was added to the standard solution (i.e., residual liquid) at time t4 to change the pH to 6.9. As a result, the absorbance was sharply increased and was then substantially constant to show termination of the decomposition of indigo carmine. The sharp increase in absorbance was caused by that indigo carmine escaped from the leuco structure.

Sulfuric acid (6.25 μL) was further added to the standard solution (i.e., residual liquid) at time t5 to change the pH to 2.4. As a result, the absorbance was gradually decreased to show decomposition of indigo carmine.

As described above, the termination and the restart of the activity can be controlled by controlling the pH of a plasma-treated liquid as in Reference Example, without being limited to second treatment liquids generated from a neutral first treatment liquid as in Examples 17 to 19. In addition, the liquid to be plasma-treated is not limited to phosphate buffer solutions and may be another liquid, such as a standard solution, and the termination and the restart of the activity can be controlled according to the pH of the liquid. Furthermore, the pH of a plasma-treated liquid can be controlled not only by an acid or base, but also by a salt.

The pH values before the termination and after the restart of the activity can be arbitrarily adjusted. Therefore, the conditions for the activity can be modified, or the activity can be performed in multiple stages.

### Modification Examples

### Modification Example 1

The gas supplied to a liquid in the generation of plasma may be a gas other than air.

FIGS. 31A and 31B show the results of a test of indigo carmine decomposition by second treatment liquids prepared by supplying various gases in the generation of plasma. FIG. 31A shows the results of decomposition by acidic second treatment liquids. FIG. 31B shows the results of decomposition by alkaline second treatment liquids.

Herein, the liquid samples were prepared as follows. A phosphate buffer solution having a pH of 8.3 or a pH of 7.2 was plasma-treated while supplying air, oxygen, nitrogen, or argon to generate a first treatment liquid. Sulfuric acid was added to this first treatment liquid to generate an acidic second treatment liquid. Alternatively, an aqueous sodium hydroxide solution was added to the first treatment liquid to generate an alkaline second treatment liquid. The explanatory notes in FIGS. 31A and 31B show the types of the gas supplied in the generation of plasma and the pH values of second treatment liquids.

As shown in FIGS. 31A and 31B, although the decomposition ability varied depending on the type of the gas, all the second treatment liquids had high decomposition ability. When the gas was air or nitrogen, the decomposition ability in acidification was high, compared to the other gases. This suggests that the plasma treatment produces nitrogen oxide-based active species, such as peroxynitrite.

### Modification Example 2

For example, plasma generator 50 may generate plasma 92 near a liquid 90. For example, at least one of the first electrode 52 and the second electrode 53 may be disposed in the air without being in contact with the liquid 90.

In this case, for example, the air on or near the surface of the liquid 90 is exposed to the plasma 92. As a result, active species are probably produces in the liquid, and nano-bubbles encapsulating the gas to which the plasma 92 was applied were probably generated. The generated nano-bubbles probably discharge active species, such as radicals, into the liquid, when the first treatment liquid was acidified or alkalinized. As a result, a second treatment liquid having an activity can be prepared.

### Modification Example 3

The pH regulator may be any material that can change pH. FIG. 32 shows the results of a test of indigo carmine decomposition by various second treatment liquids generated by acidification or alkalinization of a plasma-treated phosphate buffer solution (pH: 7) with a variety of pH regulators. The explanatory notes in FIG. 32 show the pH regulators added to a phosphate buffer solution and the pH values of the resulting second treatment liquids. As shown in FIG. 32, the second treatment liquid acidified with nitric acid and the second treatment liquid alkalinized with ammonia water had high decomposition ability. The pH regulator may be, for example, an ordinary household detergent or lemon juice.

### Modification Example 4

The object treatment apparatus 10c according to the Fifth Embodiment may further include a dilution unit for supplying a dilution liquid to the container 20c. This dilution unit may have, for example, the same structure as that of the dilution unit 70 shown in FIG. 2 and can be controlled by the control circuit 40c.

The method of treating an object according to Modification Example 4 further includes, in the flow chart shown in FIG. 24, a step of diluting the residual liquid when the residual liquid is judged to not be reused (in the case of “No” in S50). As a result, the activity of the residual liquid can be further reduced. The diluted residual liquid is, for example, discharged from the object treatment apparatus 10c and is discarded.

### Modification Example 5

In the Fifth Embodiment, a plasma-treated liquid was brought into contact with the object 11c in the container 20c, but the Embodiment is not limited thereto. For example, a plasma-treated liquid may be brought into contact with the object 11c in a container different from the container 20c, and the residual plasma-treated liquid may be placed in the container 20c through the inlet 21.

### Modification Example 6

For example, in the above-described Embodiments, the pH may be adjusted by electrolysis instead of the use of a pH regulator. For example, a container is divided into a first region and a second region by a barrier membrane, and the first region contains a plasma-treated liquid, and the second region contains a certain liquid. An electrode A is disposed in the first region, and an electrode B is disposed in the second region. In this structure, an application of a voltage between the electrode A and the electrode B electrolyzes the plasma-treated liquid. For example, in a case that a plasma-treated liquid having a pH of less than 6 is contained in the first region, the electrode A and the electrode B are used as a negative electrode and a positive electrode, respectively, and a voltage is applied such that the electrode A is negative with respect to the electrode B. As a result, the pH of the plasma-treated liquid is increased, and, for example, the electrolysis is terminated when the plasma-treated liquid is neutralized. In a case that a plasma-treated liquid having a pH of 9 or more is contained in the first region, the electrode A and the electrode B are respectively used as a positive electrode and a negative electrode, and a voltage is applied such that the electrode A is positive with respect to the electrode B. As a result, the pH of the plasma-treated liquid is decreased, and, for example, the electrolysis is terminated when the plasma-treated liquid is neutralized. The electrolysis may be applied not only in the case of neutralizing a plasma-treated liquid but also, for example, in a case of acidification. The change in pH may be monitored with, for example, the above-described pH sensor.

### Other Embodiments

The method of generating a treatment liquid, the treatment liquid generation apparatus, the method of treating an object, and the treatment liquid according to one or more aspects have been described based on the Embodiments and Modification Examples, but the present disclosure is not limited to these Embodiments and Examples. The present disclosure also encompasses embodiments provided by applying various modifications that can be conceived by those skilled in the art to the above-described Embodiments and embodiments established by combining components in different Embodiments, without departing from the gist of the present disclosure.

A liquid treatment apparatus according to an aspect of an embodiment comprises: a container that contains a liquid; a feeder that supplies a pH regulator to the container; and a control circuit that controls the feeder. The control circuit instructs the feeder to supply the pH regulator to the container, when the container contains a plasma-treated liquid having a pH of 6 or more and 9 or less, to change the pH of the plasma-treated liquid to less than 6 or to higher than 9 the plasma-treated liquid being the liquid that has been treated with plasma generated in or near the liquid.

For example, the liquid treatment apparatus may further comprise a plasma generator that generates plasma in or near the liquid, the plasma generator including a pair of electrodes and a power supply that applies a voltage to the pair of electrodes. With this configuration, the control circuit may instruct the plasma generator to generate plasma and to generate the plasma-treated liquid having a pH of 6 or more and 9 or less. The control circuit may instruct the plasma generator to generate plasma and then instruct the feeder to supply the pH regulator to the container, to generate the plasma-treated liquid having a pH of 6 or more and 9 or less. During the generation of the plasma, the control circuit may further instruct the feeder to supply the pH regulator to the container, when an average pH per unit time of the liquid is less than 6 or higher than 9, to change the pH of the liquid to 6 or more and 9 or less.

For example, the liquid treatment apparatus may further comprise: a plasma generator that generates plasma in or near the liquid, the plasma generator including a first pair of electrodes and a first power supply that applies a voltage to the first pair of electrodes; and an electrolyzer that electrolyze the liquid, the electrolyzer including a second pair of electrodes and a second power supply that applies a voltage to the second pair of electrodes. With this configuration, during the generation of the plasma, the control circuit may further instruct the electrolizer to electrolyze the liquid when an average pH per unit time of the liquid is less than 6 or higher than 9, to change the pH of the liquid to 6 or more and 9 or less.

A liquid treatment apparatus according to an aspect of an embodiment comprises: a container that contains a liquid; a first pair of electrodes; a first power supply that applies a voltage to the first pair of electrodes; and a control circuit that controls the first power supply. With this configuration, the control circuit instructs the first power supply to apply a voltage to the first pair of electrodes, when the container contains the plasma-treated liquid having a pH of 6 or more and 9 or less, to change the pH of the plasma-treated liquid to less than 6 or to higher than 9, the plasma-treated liquid being the liquid that has been treated with plasma generated in or near the liquid.

The liquid treatment apparatus may further comprise: a plasma generator that generates plasma in or near the liquid, the plasma generator including a second pair of electrodes and a second power supply that applies a voltage to the second pair of electrodes. With this configuration, the control circuit may instruct the plasma generator to generate plasma to generate the plasma-treated liquid having a pH of 6 or more and 9 or less. The control circuit may instruct the plasma generator to generate plasma and then instruct the first power supply to apply a voltage to the first pair of electrodes, to generate a plasma-treated liquid having a pH of 6 or more and 9 or less. During the generation of the plasma, the control circuit may instruct the first power supply to apply a voltage to the first electrode pair, when an average pH per unit time of the liquid is less than 6 or higher than 9, to change the pH of the liquid to 6 or more and 9 or less.

An object treatment apparatus according to an aspect of an embodiment comprises one of the above-noted liquid treatment apparatuses, wherein the control circuit further brings the plasma-treated liquid into contact with an object. For example, the control circuit may change the pH of the plasma-treated liquid to less than 6 or to higher than 9 before bringing the plasma-treated liquid into contact with the object. The control circuit may instruct the feeder to supply the pH regulator to the container in a state that the plasma-treated liquid is in contact with the object. The control circuit may instruct the first power supply to supply the voltage to the first pair of electrodes in a state that the plasma-treated liquid is in contact with the object. The control circuit may bring the plasma-treated liquid into contact with the object before changing the pH of the plasma-treated liquid to 6 or more and 9 or less.

A plasma-treated liquid according to an aspect of an embodiment is a liquid that has been treated with plasma generated in or near the liquid. This plasma-treated liquid has following characteristics (A), (B), and (C). (A) the plasma-treated liquid has a pH of 6 or more and 9 or less. (B) a decomposition rate of indigo carmine is 0.02 ppm/min or less, calculated from a change in absorbance of light having a wavelength of 610 nm, when 10 ppm of indigo carmine is added to the plasma-treated liquid at 20° C. (C) (c1) when a 4.5 N sulfuric acid solution is mixed with the plasma-treated liquid to give a pH of 2.5, the decomposition rate of indigo carmine at 10 seconds after addition of the sulfuric acid is 0.05 ppm/min or more, or (c2) when an aqueous 4.5 N sodium hydroxide solution is mixed with the plasma-treated liquid to give a pH of 11.5, the decomposition rate of indigo carmine at 10 seconds after addition of the aqueous sodium hydroxide solution is 0.1 ppm/min or more.

The above-described Embodiments can be subjected to a variety of, for example, modifications, replacements, additions, or omissions within the scope of the claims or a scope equivalent thereto.

The method of generating a treatment liquid and so on according to the present disclosure can be used in, for example, decomposition of an organic material or sterilization of microorganisms, bacteria, etc.

What is claimed is:

1. A method comprising:
preparing a plasma-treated liquid having a pH of 6 or more and 9 or less, the plasma-treated liquid being a liquid that has been treated with plasma generated in or near the liquid; and
changing the pH of the plasma-treated liquid to less than 6 or to higher than 9.

2. The method according to claim 1, wherein
the preparing of the plasma-treated liquid includes generating the plasma in or near the liquid to generate the plasma-treated liquid while adjusting or maintaining the pH of the liquid to 6 or more and 9 or less.

3. The method according to claim 1, wherein
the preparing of the plasma-treated liquid includes:
generating the plasma in or near the liquid; and
adjusting or maintaining the pH to 6 or more and 9 or less after the generating of the plasma.

4. The method according to claim 1, wherein
in the changing of the pH of the plasma-treated liquid, (i) an acid, base, or salt; (ii) a solution containing at least one of acids, bases, and salts; (iii) a gas or solid that is dissolvable in the plasma-treated liquid to show acidity or basicity; (iv) a solution containing a microorganism producing the gas or solid is added to the plasma-treated liquid.

5. The method according to claim 1, wherein
in the changing of the pH of the plasma-treated liquid, the pH of the plasma-treated liquid is changed to less than 3.5 or to higher than 10.5.

6. The method according to claim 1, further comprising:
diluting the plasma-treated liquid, before the changing of the pH of the plasma-treated liquid.

7. The method according to claim 1, wherein
in the preparing of the plasma-treated liquid, the plasma-treated liquid is electrolyzed.

8. The method according to claim 1, wherein
in the changing of the pH of the plasma-treated liquid, the plasma-treated liquid is electrolyzed.

9. The method according to claim 1, further comprising:
bringing the plasma-treated liquid into contact with an object to be treated.

10. The method according to claim 9, further comprising:
changing the pH of the plasma-treated liquid, before the bringing of the plasma-treated liquid into contact with the object.

11. The method according to claim 9, wherein
the bringing of the plasma-treated liquid into contact with the object and the changing of the pH of the plasma-treated liquid are concurrently performed.

12. The method according to claim 9, further comprising:
changing the pH of the plasma-treated liquid to 6 or more and 9 or less, after the bringing of the plasma-treated liquid into contact with the object.

13. The method according to claim 12, wherein
in the changing of the pH of the plasma-treated liquid to 6 or more and 9 or less, a solution containing an acid, base, or salt is added to the plasma-treated liquid.

14. The method according to claim 12, wherein
in the changing of the pH of the plasma-treated liquid to 6 or more and 9 or less, the plasma-treated liquid is electrolyzed.
